SEQUENCE LISTING (43 sequences)

SEQ ID NO: 1 - protein, 691 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFE

SEQ ID NO: 2 - protein, 1452 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYGSGATNFSLLKQAGDVEENPGPTVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDIAQKDEVTKKDEASPLFA

SEQ ID NO: 3 - protein, 1452 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDIAQKDEVTKKDEASPLFAGSGATNFSLLKQAGDVEENPGPTVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAY

SEQ ID NO: 4 - protein, 572 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAY

SEQ ID NO: 5 - protein, 831 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIW

SEQ ID NO: 6 - protein, 572 aa, synthetic construct; identical to SEQ ID NO: 4

SEQ ID NO: 7 - protein, 858 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDIAQKDEVTKKDEASPLFA

SEQ ID NO: 8 - protein, 572 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTAASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAY

SEQ ID NO: 9 - protein, 572 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTAASSSTHKGVVVWREGPRWEIKEIADLGASVQQLAARAVAMALLLWPTTPTNVVTASAFVAKMLLKMGQEGVPSTAAAFILEDALSQRSAMAAVLHVRSHSEVPGFFTEGNDVADSQATFQAY

SEQ ID NO: 10 - protein, 314 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERW

SEQ ID NO: 11 - protein, 422 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSI

SEQ ID NO: 12 - protein, 449 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFT

SEQ ID NO: 13 - protein, 449 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAET

SEQ ID NO: 14 - protein, 572 aa, synthetic construct
TVALHLAIPLKWKPDHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLVVGQVLEPLRLKHPSLCMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVRLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALKGFAGKIRSSDTPSIFDIARPLHVSLKVRVTDHPVPGPTVFTDASSSTHKGVVVWREGPRWEIKEIADLGASVQQLEARAVAMALLLWPTTPTNVVTNSAFVAKMLLKMGQEGVPSTAAAFILEDALSQRSAMAAVLHVRSHSEVPGFFTEGNDVADSQATFQAY

SEQ ID NO: 15 - protein, 427 aa, synthetic construct
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 16 - protein, 560 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 17 - protein, 439 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAET

SEQ ID NO: 18 - protein, 455 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTAASSST

SEQ ID NO: 19 - protein, 489 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTAASSSTHKGVVVWREGPRWEIKEIADLGASVQQLAARAVA

SEQ ID NO: 20 - protein, 523 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTAASSSTHKGVVVWREGPRWEIKEIADLGASVQQLAARAVAMALLLWPTTPTNVVTASAFVAKMLLKMGQEGVPS

SEQ ID NO: 21 - DNA, 2094 bp, synthetic construct
  source 1..2094: mol_type = other DNA
accctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtc

SEQ ID NO: 22 - DNA, 4386 bp, synthetic construct
  source 1..4386: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagatgcttcatcttcaacccacaaaggcgtagtggtttggcgagagggccccaggtgggagataaaagaaatagccgacctcggggcaagcgttcaacagcttgaggccagagcagtggctatggccttgctgctgtggcctactactcccaccaatgttgtgacagattctgccttcgtcgcaaagatgctgctgaagatgggccaagagggcgttccttcaaccgctgctgcatttattctcgaggatgctcttagccaaaggtccgccatggcagccgttcttcacgtgcggtcacattctgaagtgccaggattcttcactgagggcaatgatgtcgcagactcccaagcaaccttccaggcatatggcagcggcgcaactaacttttccttgttgaagcaggctggcgatgtagaggagaatcctggtccaactgttgcgctacatctggctattccgctcaaatggaagccaaaccacacgcctgtgtggattgaccagtggccccttcctgaaggtaaacttgtagcgctaacgcaattagtggaaaaagaattacagttaggacatatagaaccttcacttagttgctggaacacacctgtctttgtgatccggaaggcttccgggtcttatcgcttattgcatgacttgcgcgctgttaacgctaagcttgttccttttggggccgtccaacagggggcgccggttctctccgcgctcccgcgtggctggcccctgatggtcctagacctcaaggattgcttcttttctattcctcttgcggaacaagatcgcgaagcttttgcatttacgctcccctctgtgaataaccaggcccccgctcgaagattccaatggaaggtcttgccccaagggatgacctgttctcccactatctgtcagttgatagtgggtcaaatacttgagcccttgcgactcaagcacccatctctgcgcatgttgcattatatggatgatcttttgctagccgcctcaagtcatgatgggttggaagcggcaggggaggaggttatcagtacattggaaagagccgggttcaccatttcgcctgataaggtccagagggagcccggagtacaatatcttgggtacaagttaggcagtacgtatgtagcacccgtaggcctggtagcagaacccaggatagccaccttgtgggatgttcagaagctggtggggtcacttcagtggcttcgcccagcgctaggaatcccgcctcgactgatgggccccttttatgagcagttacgagggtcagatcctaacgaggcgagggaatggaatctagacatgaaaatggcctggagagagatcgtgcagctcagcaccactgctgccttggaacgatgggaccctgccctgcctctggaaggagcggtcgctagatgtgaacagggggcaataggggtcctgggacagggactgtccacacacccaaggccatgtttgtggttattctccacccaacccaccaaggcgtttactgcttggttagaagtgctcacccttttgattactaagctacgtgcttcggcagtgcgaacctttggcaaggaggttgatatcctcctgttgcctgcatgctttcgggaggaccttccgctcccggaggggatcctgttagcccttagggggtttgcaggaaaaatcaggagtagtgacacgccatctatttttgacattgcgcgtccactgcatgtttctctgaaagtgagggttaccgaccaccctgtaccgggacccactgtctttaccgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactgactctgcgtttgttgcgaaaatgttactcaagatggggcaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccacctttcaagcgtatcccttgagagaggctaaagatctccataccgctctccatatcggaccccgcgcgctatccaaagcgtgtaatatatctatgcagcaggctagggaggttgttcagacctgcccgcattgtaattcagcccctgcgttggaggccggggtaaaccctaggggtttgggacccctacagatatggcagacagactttacactagagcctagaatggctccccgttcctggctcgctgttactgtggataccgcctcatctgcgatagtcgtaactcagcatggccgtgtcacatcggttgctgcacaacatcattgggccacggctatcgccgttttgggaagaccaaaggccataaaaacagataatgggtcctgcttcacgtctaaatccacgcgagagtggctcgcgagatgggggatagcacacaccaccgggattccgggtaattcccagggtcaagctatggtagagcgggccaaccggctcctgaaagataagatccgtgtgcttgcggagggggatggctttatgaaaagaatccccaccagcaaacagggggaactattagccaaggcaatgtatgcccttaatcactttgagcgtggtgaaaacacaaaaacaccgatacaaaaacactggagacctaccgttcttacagaaggacccccggttaaaatacgaatagagacaggggagtgggaaaaaggatggaacgtgctggtctggggacgaggttatgcagctgtgaaaaacagggacactgataaggttatttgggtaccctctcgaaaagttaaaccggacatcgcccaaaaggatgaggtgactaagaaagatgaggcgagccctctctttgcagaattcgagcccaagaagaagaggaaagtc

SEQ ID NO: 23 - DNA, 4386 bp, synthetic construct
  source 1..4386: mol_type = other DNA
actgttgcgctacatctggctattccgctcaaatggaagccaaaccacacgcctgtgtggattgaccagtggccccttcctgaaggtaaacttgtagcgctaacgcaattagtggaaaaagaattacagttaggacatatagaaccttcacttagttgctggaacacacctgtctttgtgatccggaaggcttccgggtcttatcgcttattgcatgacttgcgcgctgttaacgctaagcttgttccttttggggccgtccaacagggggcgccggttctctccgcgctcccgcgtggctggcccctgatggtcctagacctcaaggattgcttcttttctattcctcttgcggaacaagatcgcgaagcttttgcatttacgctcccctctgtgaataaccaggcccccgctcgaagattccaatggaaggtcttgccccaagggatgacctgttctcccactatctgtcagttgatagtgggtcaaatacttgagcccttgcgactcaagcacccatctctgcgcatgttgcattatatggatgatcttttgctagccgcctcaagtcatgatgggttggaagcggcaggggaggaggttatcagtacattggaaagagccgggttcaccatttcgcctgataaggtccagagggagcccggagtacaatatcttgggtacaagttaggcagtacgtatgtagcacccgtaggcctggtagcagaacccaggatagccaccttgtgggatgttcagaagctggtggggtcacttcagtggcttcgcccagcgctaggaatcccgcctcgactgatgggccccttttatgagcagttacgagggtcagatcctaacgaggcgagggaatggaatctagacatgaaaatggcctggagagagatcgtgcagctcagcaccactgctgccttggaacgatgggaccctgccctgcctctggaaggagcggtcgctagatgtgaacagggggcaataggggtcctgggacagggactgtccacacacccaaggccatgtttgtggttattctccacccaacccaccaaggcgtttactgcttggttagaagtgctcacccttttgattactaagctacgtgcttcggcagtgcgaacctttggcaaggaggttgatatcctcctgttgcctgcatgctttcgggaggaccttccgctcccggaggggatcctgttagcccttagggggtttgcaggaaaaatcaggagtagtgacacgccatctatttttgacattgcgcgtccactgcatgtttctctgaaagtgagggttaccgaccaccctgtaccgggacccactgtctttaccgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactgactctgcgtttgttgcgaaaatgttactcaagatggggcaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccacctttcaagcgtatcccttgagagaggctaaagatctccataccgctctccatatcggaccccgcgcgctatccaaagcgtgtaatatatctatgcagcaggctagggaggttgttcagacctgcccgcattgtaattcagcccctgcgttggaggccggggtaaaccctaggggtttgggacccctacagatatggcagacagactttacactagagcctagaatggctccccgttcctggctcgctgttactgtggataccgcctcatctgcgatagtcgtaactcagcatggccgtgtcacatcggttgctgcacaacatcattgggccacggctatcgccgttttgggaagaccaaaggccataaaaacagataatgggtcctgcttcacgtctaaatccacgcgagagtggctcgcgagatgggggatagcacacaccaccgggattccgggtaattcccagggtcaagctatggtagagcgggccaaccggctcctgaaagataagatccgtgtgcttgcggagggggatggctttatgaaaagaatccccaccagcaaacagggggaactattagccaaggcaatgtatgcccttaatcactttgagcgtggtgaaaacacaaaaacaccgatacaaaaacactggagacctaccgttcttacagaaggacccccggttaaaatacgaatagagacaggggagtgggaaaaaggatggaacgtgctggtctggggacgaggttatgcagctgtgaaaaacagggacactgataaggttatttgggtaccctctcgaaaagttaaaccggacatcgcccaaaaggatgaggtgactaagaaagatgaggcgagccctctctttgcaggctctggagccacaaatttcagtttgctcaagcaggctggagacgtcgaagagaatcccgggccaactgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagatgcttcatcttcaacccacaaaggcgtagtggtttggcgagagggccccaggtgggagataaaagaaatagccgacctcggggcaagcgttcaacagcttgaggccagagcagtggctatggccttgctgctgtggcctactactcccaccaatgttgtgacagattctgccttcgtcgcaaagatgctgctgaagatgggccaagagggcgttccttcaaccgctgctgcatttattctcgaggatgctcttagccaaaggtccgccatggcagccgttcttcacgtgcggtcacattctgaagtgccaggattcttcactgagggcaatgatgtcgcagactcccaagcaaccttccaggcatatgaattcgagcccaagaagaagaggaaagtc

SEQ ID NO: 24 - DNA, 1746 bp, synthetic construct
  source 1..1746: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagatgcttcatcttcaacccacaaaggcgtagtggtttggcgagagggccccaggtgggagataaaagaaatagccgacctcggggcaagcgttcaacagcttgaggccagagcagtggctatggccttgctgctgtggcctactactcccaccaatgttgtgacagattctgccttcgtcgcaaagatgttgctgaagatgggccaagagggcgttccttcaaccgctgctgcatttattctcgaggatgctcttagccaaaggtccgccatggcagccgttcttcacgtgcggtcacattctgaagtgccaggattcttcactgagggcaatgatgtcgcagactcccaagcaaccttccaggcatatgaattcgagcccaagaagaagaggaaagtc

SEQ ID NO: 25 - DNA, 2604 bp, synthetic construct
  source 1..2604: mol_type = other DNA
actgttgcgctacatctggctattccgctcaaatggaagccaaaccacacgcctgtgtggattgaccagtggccccttcctgaaggtaaacttgtagcgctaacgcaattagtggaaaaagaattacagttaggacatatagaaccttcacttagttgctggaacacacctgtctttgtgatccggaaggcttccgggtcttatcgcttattgcatgacttgcgcgctgttaacgctaagcttgttccttttggggccgtccaacagggggcgccggttctctccgcgctcccgcgtggctggcccctgatggtcctagacctcaaggattgcttcttttctattcctcttgcggaacaagatcgcgaagcttttgcatttacgctcccctctgtgaataaccaggcccccgctcgaagattccaatggaaggtcttgccccaagggatgacctgttctcccactatctgtcagttgatagtgggtcaaatacttgagcccttgcgactcaagcacccatctctgcgcatgttgcattatatggatgatcttttgctagccgcctcaagtcatgatgggttggaagcggcaggggaggaggttatcagtacattggaaagagccgggttcaccatttcgcctgataaggtccagagggagcccggagtacaatatcttgggtacaagttaggcagtacgtatgtagcacccgtaggcctggtagcagaacccaggatagccaccttgtgggatgttcagaagctggtggggtcacttcagtggcttcgcccagcgctaggaatcccgcctcgactgatgggccccttttatgagcagttacgagggtcagatcctaacgaggcgagggaatggaatctagacatgaaaatggcctggagagagatcgtgcagctcagcaccactgctgccttggaacgatgggaccctgccctgcctctggaaggagcggtcgctagatgtgaacagggggcaataggggtcctgggacagggactgtccacacacccaaggccatgtttgtggttattctccacccaacccaccaaggcgtttactgcttggttagaagtgctcacccttttgattactaagctacgtgcttcggcagtgcgaacctttggcaaggaggttgatatcctcctgttgcctgcatgctttcgggaggaccttccgctcccggaggggatcctgttagcccttagggggtttgcaggaaaaatcaggagtagtgacacgccatctatttttgacattgcgcgtccactgcatgtttctctgaaagtgagggttaccgaccaccctgtaccgggacccactgtctttaccgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactgactctgcgtttgttgcgaaaatgttactcaagatggggcaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccacctttcaagcgtatcccttgagagaggctaaagatctccataccgctctccatatcggaccccgcgcgctatccaaagcgtgtaatatatctatgcagcaggctagggaggttgttcagacctgcccgcattgtaattcagcccctgcgttggaggccggggtaaaccctaggggtttgggacccctacagatatggcagacagactttacactagagcctagaatggctccccgttcctggctcgctgttactgtggataccgcctcatctgcgatagtcgtaactcagcatggccgtgtcacatcggttgctgcacaacatcattgggccacggctatcgccgttttgggaagaccaaaggccataaaaacagataatgggtcctgcttcacgtctaaatccacgcgagagtggctcgcgagatgggggatagcacacaccaccgggattccgggtaattcccagggtcaagctatggtagagcgggccaaccggctcctgaaagataagatccgtgtgcttgcggagggggatggctttatgaaaagaatccccaccagcaaacagggggaactattagccaaggcaatgtatgcccttaatcactttgagcgtggtgaaaacacaaaaacaccgatacaaaaacactggagacctaccgttcttacagaaggacccccggttaaaatacgaatagagacaggggagtgggaaaaaggatggaacgtgctggtctggggacgaggttatgcagctgtgaaaaacagggacactgataaggttatttgggtaccctctcgaaaagttaaaccggacatcgcccaaaaggatgaggtgactaagaaagatgaggcgagccctctctttgcagaattcgagcccaagaagaagaggaaagtc

SEQ ID NO: 26 - DNA, 1716 bp, synthetic construct
  source 1..1716: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagatgcttcatcttcaacccacaaaggcgtagtggtttggcgagagggccccaggtgggagataaaagaaatagccgacctcggggcaagcgttcaacagcttgaggccagagcagtggctatggccttgctgctgtggcctactactcccaccaatgttgtgacagattctgccttcgtcgcaaagatgttgctgaagatgggccaagagggcgttccttcaaccgctgctgcatttattctcgaggatgctcttagccaaaggtccgccatggcagccgttcttcacgtgcggtcacattctgaagtgccaggattcttcactgagggcaatgatgtcgcagactcccaagcaaccttccaggcatat

SEQ ID NO: 27 - DNA, 2574 bp, synthetic construct
  source 1..2574: mol_type = other DNA
actgttgcgctacatctggctattccgctcaaatggaagccaaaccacacgcctgtgtggattgaccagtggccccttcctgaaggtaaacttgtagcgctaacgcaattagtggaaaaagaattacagttaggacatatagaaccttcacttagttgctggaacacacctgtctttgtgatccggaaggcttccgggtcttatcgcttattgcatgacttgcgcgctgttaacgctaagcttgttccttttggggccgtccaacagggggcgccggttctctccgcgctcccgcgtggctggcccctgatggtcctagacctcaaggattgcttcttttctattcctcttgcggaacaagatcgcgaagcttttgcatttacgctcccctctgtgaataaccaggcccccgctcgaagattccaatggaaggtcttgccccaagggatgacctgttctcccactatctgtcagttgatagtgggtcaaatacttgagcccttgcgactcaagcacccatctctgcgcatgttgcattatatggatgatcttttgctagccgcctcaagtcatgatgggttggaagcggcaggggaggaggttatcagtacattggaaagagccgggttcaccatttcgcctgataaggtccagagggagcccggagtacaatatcttgggtacaagttaggcagtacgtatgtagcacccgtaggcctggtagcagaacccaggatagccaccttgtgggatgttcagaagctggtggggtcacttcagtggcttcgcccagcgctaggaatcccgcctcgactgatgggccccttttatgagcagttacgagggtcagatcctaacgaggcgagggaatggaatctagacatgaaaatggcctggagagagatcgtgcagctcagcaccactgctgccttggaacgatgggaccctgccctgcctctggaaggagcggtcgctagatgtgaacagggggcaataggggtcctgggacagggactgtccacacacccaaggccatgtttgtggttattctccacccaacccaccaaggcgtttactgcttggttagaagtgctcacccttttgattactaagctacgtgcttcggcagtgcgaacctttggcaaggaggttgatatcctcctgttgcctgcatgctttcgggaggaccttccgctcccggaggggatcctgttagcccttagggggtttgcaggaaaaatcaggagtagtgacacgccatctatttttgacattgcgcgtccactgcatgtttctctgaaagtgagggttaccgaccaccctgtaccgggacccactgtctttaccgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactgactctgcgtttgttgcgaaaatgttactcaagatggggcaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccacctttcaagcgtatcccttgagagaggctaaagatctccataccgctctccatatcggaccccgcgcgctatccaaagcgtgtaatatatctatgcagcaggctagggaggttgttcagacctgcccgcattgtaattcagcccctgcgttggaggccggggtaaaccctaggggtttgggacccctacagatatggcagacagactttacactagagcctagaatggctccccgttcctggctcgctgttactgtggataccgcctcatctgcgatagtcgtaactcagcatggccgtgtcacatcggttgctgcacaacatcattgggccacggctatcgccgttttgggaagaccaaaggccataaaaacagataatgggtcctgcttcacgtctaaatccacgcgagagtggctcgcgagatgggggatagcacacaccaccgggattccgggtaattcccagggtcaagctatggtagagcgggccaaccggctcctgaaagataagatccgtgtgcttgcggagggggatggctttatgaaaagaatccccaccagcaaacagggggaactattagccaaggcaatgtatgcccttaatcactttgagcgtggtgaaaacacaaaaacaccgatacaaaaacactggagacctaccgttcttacagaaggacccccggttaaaatacgaatagagacaggggagtgggaaaaaggatggaacgtgctggtctggggacgaggttatgcagctgtgaaaaacagggacactgataaggttatttgggtaccctctcgaaaagttaaaccggacatcgcccaaaaggatgaggtgactaagaaagatgaggcgagccctctctttgca

SEQ ID NO: 28 - DNA, 1746 bp, synthetic construct
  source 1..1746: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagctgcttcatcttcaacccacaaaggcgtagtggtttggcgagagggccccaggtgggagataaaagaaatagccgacctcggggcaagcgttcaacagcttgaggccagagcagtggctatggccttgctgctgtggcctactactcccaccaatgttgtgacagattctgccttcgtcgcaaagatgttgctgaagatgggccaagagggcgttccttcaaccgctgctgcatttattctcgaggatgctcttagccaaaggtccgccatggcagccgttcttcacgtgcggtcacattctgaagtgccaggattcttcactgagggcaatgatgtcgcagactcccaagcaaccttccaggcatatgaattcgagcccaagaagaagaggaaagtc

SEQ ID NO: 29 - DNA, 1746 bp, synthetic construct
  source 1..1746: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagctgcttcatcttcaacccacaaaggcgtagtggtttggcgagagggccccaggtgggagataaaagaaatagccgacctcggggcaagcgttcaacagcttgcggccagagcagtggctatggccttgctgctgtggcctactactcccaccaatgttgtgacagcttctgccttcgtcgcaaagatgttgctgaagatgggccaagagggcgttccttcaaccgctgctgcatttattctcgaggatgctcttagccaaaggtccgccatggcagccgttcttcacgtgcggtcacattctgaagtgccaggattcttcactgagggcaatgatgtcgcagactcccaagcaaccttccaggcatatgaattcgagcccaagaagaagaggaaagtc

SEQ ID NO: 30 - DNA, 972 bp, synthetic construct
  source 1..972: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaattcgagcccaagaagaagaggaaagtc

SEQ ID NO: 31 - DNA, 1296 bp, synthetic construct
  source 1..1296: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcgaattcgagcccaagaagaagaggaaagtc

SEQ ID NO: 32 - DNA, 1377 bp, synthetic construct
  source 1..1377: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagaattcgagcccaagaagaagaggaaagtc

SEQ ID NO: 33 - DNA, 1380 bp, synthetic construct
  source 1..1380: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttcgaattcgagcccaagaagaagaggaaagtc

SEQ ID NO: 34 - DNA, 1746 bp, synthetic construct
  source 1..1746: mol_type = other DNA
actgttgcgctacatctggctattccgctcaaatggaagccagaccacacgcctgtgtggattgaccagtggcccctccctgaaggtaaacttgtagcgctaacgcaattagtggaaaaagaattacagttaggacatatagaaccttcacttagttgttggaacacacctgtcttcgtgatccggaaggcttccgggtcttaccgcttactgcatgatttgcgcgctgttaacgccaagcttgttccttttggggccgtccaacagggggcgccagttctctccgcgctcccgcgtggctggcccctgatggtcttagacctcaaggattgcttcttttctatccctcttgcggaacaagatcgcgaagcttttgcatttacgctcccctctgtgaataaccaggcccccgctcgaagattccaatggaaggtcttgccccaagggatgacctgttctcccactatctgtcagttggtagtgggtcaggtacttgagcccttgcgactcaagcacccatctctgtgcatgttgcattatatggatgatcttttgctagccgcctcaagtcacgatgggttggaagcggcaggggaggaggttatcagtacattggaaagagccgggttcactatttcgcctgataaggtccagagggagcccggagtacaatatcttgggtacaagttaggcagtacgtatgtagcacccgtaggcctggtagcagaacccaggatagccaccttgtgggatgttcaaaagctggtggggtcacttcagtggcttcgcccagcgttaggaatcccgccacgactgatgggccccttctatgagcagttacgagggtcagatcctaacgaggcgagggaatggaatctagacatgaaaatggcctggagagagatcgtacggcttagcaccactgctgccttggaacgatgggaccctgccctgcctctggaaggagcggtcgctagatgtgaacagggggcaataggggttttgggacagggactgtccacacacccaaggccatgcttgtggttattctccacccaacccaccaaggcgtttactgcttggttagaagtgctcacccttttgattactaagctacgtgcttcggcagtgcgaacctttggcaaggaggtcgatatcctcctgttgcctgcatgctttcgggaggaccttccgctcccagaggggatcctgttagcccttaaggggtttgcaggaaaaatcaggagtagtgacacgccatctatttttgacattgcgcgtccactgcatgtttctctgaaagtgagggttaccgaccaccctgtgccgggacccactgtctttactgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactaactccgcgtttgttgcgaaaatgttactcaagatgggacaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccaccttccaagcgtatgaattcgagcccaagaagaagaggaaagtc

SEQ ID NO: 35 - DNA, 1347 bp, synthetic construct
  source 1..1347: mol_type = other DNA
atggataccagcaatctgatggaacaaattctgagcagcgataatctgaatcgtgcatatctgcaggttgtgcgtaataaaggtgccgaaggtgttgatggtatgaaatataccgaactgaaagaacacctggccaaaaatggtgaaaccattaaaggtcagctgcgtacccgtaaatacaaaccgcagcctgcacgtcgtgttgaaattccgaaaccggatggtggtgttcgtaatctgggtgttccgaccgttaccgatcgttttattcagcaggcaattgcacaggttctgaccccgatttatgaagaacagtttcatgatcacagctatggctttcgtccgaatcgttgtgcacagcaggccattctgaccgcactgaatattatgaatgatggcaatgattggatcgtggatatcgacctggaaaaattctttgataccgtgaaccacgataaactgatgaccctgattggtcgtacaatcaaagatggtgatgtgattagcatcgtgcgtaaatatctggttagcggcattatgatcgatgatgaatatgaggatagcattgttggtacaccgcagggtggtaatctgagtccgctgctggccaatattatgctgaatgaactggacaaagagatggaaaaacgcggtctgaattttgttcgttatgccgatgattgcattattatggttggtagcgaaatgagcgcaaatcgtgttatgcgtaatattagccgcttcatcgaagaaaaactgggcctgaaagttaacatgaccaaaagcaaagttgatcgtccgagtggtctgaaatacctgggttttggtttctattttgatccgcgtgcacaccagtttaaagcaaaaccgcatgcaaaaagcgtggccaaattcaaaaaacgcatgaaagaactgacctgtcgtagctggggtgttagcaatagctataaagtggaaaaactgaaccagctgattcgcggttggatcaactatttcaaaattggcagcatgaaaaccctgtgcaaagaactggatagccgtattcgttatcgtctgcgtatgtgtatttggaaacagtggaaaacaccgcagaatcaagaaaaaaatctggtgaaactgggcattgatcgtaataccgcacgtcgcgttgcatataccggtaaacgtattgcatatgtgtgcaataaaggcgcagttaatgttgccattagcaataaacgtctggcaagctttggtctgattagtatgctggattactacatcgaaaaatgcgtgacctgcccctctggcggctcaaaaagaaccgccgacggcagcgaattcgagcccaagaagaagaggaaagtc

SEQ ID NO: 36 - DNA, 1743 bp, synthetic construct
  source 1..1743: mol_type = other DNA
cccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatctggcggctcaaaaagaaccgccgacggcagcgaattcgagcccaagaagaagaggaaagtc

SEQ ID NO: 37 - DNA, 1380 bp, synthetic construct
  source 1..1380: mol_type = other DNA
cccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaacctctggcggctcaaaaagaaccgccgacggcagcgaattcgagcccaagaagaagaggaaagtc

SEQ ID NO: 38 - DNA, 1428 bp, synthetic construct
  source 1..1428: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagctgcttcatcttcaacctctggcggctcaaaaagaaccgccgacggcagcgaattcgagcccaagaagaagaggaaagtc

SEQ ID NO: 39 - DNA, 1530 bp, synthetic construct
  source 1..1530: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagctgcttcatcttcaacccacaaaggcgtagtggtttggcgagagggccccaggtgggagataaaagaaatagccgacctcggggcaagcgttcaacagcttgcggccagagcagtggcttctggcggctcaaaaagaaccgccgacggcagcgaattcgagcccaagaagaagaggaaagtc

SEQ ID NO: 40 - DNA, 1632 bp, synthetic construct
  source 1..1632: mol_type = other DNA
actgtggctctgcatctggccatcccccttaagtggaaacccaatcatacaccagtgtggattgaccaatggcccctccccgaggggaagctggtagccctgacacagctggtcgagaaggagctccagctcgggcatatcgaaccctctttgtcctgttggaacacacctgtctttgtcattagaaaggcaagtggctcctatcgcctcttgcacgatttgagagccgtgaacgccaaactcgtacccttcggtgcagtgcagcaaggtgcccccgtattgagcgctcttcctcgcggatggcctctgatggtactcgacctcaaagactgttttttcagtatacccctcgccgagcaagacagagaggcattcgccttcaccttgccatccgtaaataatcaggccccagcccggaggttccaatggaaagtcctgcctcagggtatgacttgcagcccaacaatttgccagcttattgtgggacaaatccttgaacccttgagacttaaacacccaagtctcaggatgctccactacatggacgacctgcttcttgctgcatccagccatgatggacttgaggccgccggcgaagaagttatatcaactttggaacgcgctggattcactatatcaccagataaagtacagcgggagcctggggtgcagtatctgggttacaagctcggctccacatatgtcgcccctgtcggactggtggccgagcctagaatcgcaacactgtgggacgttcaaaaattggtagggtcactgcaatggttgagacctgccctcggcattcccccccggttgatgggaccattttacgaacaattgcggggaagcgatccaaacgaagcacgagagtggaatctggatatgaaaatggcctggcgcgaaattgtacagctgagtacaacagcagcattggaacgatgggaccccgctctgcctcttgaaggagctgtggcccgatgcgagcagggagctatcggtgtccttggccaagggttgagcactcaccctcgaccatgtttgtggttgtttagcactcagcctactaaagcatttaccgcctggctcgaggtcctcacattgttgataaccaaacttcgcgcaagcgcagtgcggaccttcggaaaagaagtcgacattctgttgctccctgcctgtttcagggaagaccttccattgcccgagggcatattgctggccctcagaggcttcgctggaaagatccgaagttcagacaccccatccatcttcgatatagcacgccctctgcatgtatcccttaaagtgagggtaactgaccaccctgtacccggacccaccgtcttcacagctgcttcatcttcaacccacaaaggcgtagtggtttggcgagagggccccaggtgggagataaaagaaatagccgacctcggggcaagcgttcaacagcttgcggccagagcagtggctatggccttgctgctgtggcctactactcccaccaatgttgtgacagcttctgccttcgtcgcaaagatgttgctgaagatgggccaagagggcgttccttcatctggcggctcaaaaagaaccgccgacggcagcgaattcgagcccaagaagaagaggaaagtc

SEQ ID NO: 41 - protein, 16 aa, synthetic construct
KRPAATKKAGQAKKKK

SEQ ID NO: 42 - protein, 25 aa, synthetic construct
MSRRRKANPTKLSENAKKLAKEVEN

SEQ ID NO: 43 - protein, 18 aa, synthetic construct
KRTADGSEFESPKKKRKV

(19) United States
(12) Patent Application Publication
(10) Pub. No.: US 2025/0171810 A1
(43) Pub. Date: May 29, 2025

(54) COMPOSITIONS, SYSTEMS, AND METHODS FOR PRIME EDITING

(71) Applicant: The Trustees of Columbia University in the City of New York, New York, NY (US)

(72) Inventors: Peter M.J. Quinn, New York, NY (US); Yi-Ting Tsai, New York, NY (US); Bruna Lopes da Costa, New York, NY (US); Stephen H. Tsang, New York, NY (US)

(21) Appl. No.: 19/036,757

(22) Filed: Jan. 24, 2025

Related U.S. Application Data
(63) Continuation of application No. PCT/US2023/071132, filed on Jul. 27, 2023.
(60) Provisional application No. 63/492,886, filed on Mar. 29, 2023.
(60) Provisional application No. 63/369,558, filed on Jul. 27, 2022.

(51) Int. Cl. C12N 15/90; C12N 9/12; C12N 9/22; C12N 15/11

(52) U.S. Cl. CPC C12N 15/907; C12N 9/1276; C12N 9/22; C12N 15/11; C12Y 207/07049; C12N 2310/20

(57) ABSTRACT

The present disclosure provides systems, methods, and compositions for modifying a target nucleic acid. Particularly, the present invention relates to a polypeptide comprising a single subunit of a reverse transcriptase and a sequence-specific nuclease for use in prime-editing modification of a nucleic acid.

### STATEMENT REGARDING FEDERALLY SPONSORED RESEARCH OR DEVELOPMENT

This invention was made with government support under EY078213, EY024698, EY027285, and EY028758 awarded by the National Institutes of Health. The government has certain rights in the invention.

### FIELD

The present invention relates to systems, methods, and compositions for modifying a target nucleic acid. Particularly, the present invention relates to a polypeptide comprising a single subunit of a reverse transcriptase and a sequence-specific nuclease for use in prime-editing modification of a nucleic acid.

### CROSS-REFERENCE TO RELATED APPLICATIONS

This application is a continuation of PCT International Application No. PCT/US2023/071132, filed Jul. 27, 2023, which claims the benefit of U.S. Provisional Application Nos. 63/369,558, filed Jul. 27, 2022, and 63/492,886, filed Mar. 29, 2023, the contents of which are herein incorporated by reference in their entirety.

### SEQUENCE LISTING STATEMENT

The contents of the electronic sequence listing titled COLUM-40989.601.xml (Size: 89,575 bytes; and Date of Creation: Jul. 26, 2023) is herein incorporated by reference in its entirety.

### BACKGROUND

Clustered regularly interspaced short palindromic repeats (CRISPR)-Cas systems are powerful gene editing tools. Most CRISPR-Cas systems rely on a molecular complex that couples a guide RNA with an enzyme, Cas9, that cuts both strands of DNA thereby allowing a cell's repair machinery to introduce or delete nucleotides. These double strand breaks, however, can result in unwanted off-target effects and DNA modifications, and even cell death or lethality of the organism.

Base editing is a CRISPR-Cas9-based genome editing technology that allows the introduction of point mutations in the DNA without cutting both strands of DNA. However, canonical base editors can only create a subset of changes (C—>T, G->A, A->G, and T->C) and are less precise, resulting in the undesired introduction of mutations within an editing window of the target nucleic acid. Prime editing, similar to base editing, allows template free insertion, deletion, or nucleotide substitution without utilizing a double strand break by exploiting a reverse transcriptase fused to a Cas9 nickase. Unlike base editing, prime editing can facilitate all twelve possible transition and transversion mutations, as well as small insertion or deletion mutations. Currently, these prime editors are too large for packaging in a single adeno-associated viral vector for delivery to cells and require multi-vector delivery strategies for use. Thus, the development of components for use in safe and efficient delivery systems is crucial for the success of prime editing in the clinic.

### SUMMARY

Provided herein are polypeptides comprising: a single subunit of a multi-subunit reverse transcriptase (RNA-dependent DNA polymerases), or a variant or fragment thereof, linked to a sequence-specific nuclease, or a variant or active fragment thereof.

In some embodiments, the single subunit of a multi-subunit reverse transcriptase, or a variant or fragment thereof comprises less than 800 (e.g., less than about 750, less than about 700, less than about 650, less than about 600, less than about 550, less than about 500) amino acids.

In some embodiments, the single subunit of a multi-subunit reverse transcriptase, or a variant or fragment thereof comprises an RNaseH domain. In some embodiments, the RNaseH domain is partially or completely inactive or removed.

In some embodiments, the single subunit of a multi-subunit reverse transcriptase, or a variant or fragment thereof comprises a connection subdomain.

In some embodiments, the single subunit of a multi-subunit reverse transcriptase, or a variant or fragment thereof is derived from: avian myeloblastosis virus reverse transcriptase (AMV RT)-alpha subunit, Rous sarcoma virus Transcriptase (RSV RT)-alpha subunit, or HIV-1 reverse transcriptase (RT) p66 subunit. In some embodiments, the single subunit of a multi-subunit reverse transcriptase, or a variant or fragment thereof comprises an amino acid sequence having at least 70% (e.g., at least about 75%, at least about 80%, at least about 85%, at least about 90%, at least about 95%, at least about 97%, at least about 98%, at least about 99%, or 100%) identity to any of SEQ ID NOs: 4, 8, 9, 14, or 16.

In some embodiments, the sequence-specific nuclease is a Cas protein. In some embodiments, the Cas protein is Cas9 or a variant or fragment thereof. In some embodiments, the Cas protein is a Cas9 nickase.

In some embodiments, the single subunit of a multi-subunit reverse transcriptase, or a variant or fragment thereof is linked to the C terminus of the sequence-specific nuclease, or a variant or active fragment thereof. In some embodiments, the polypeptide further comprises a linker between the single subunit of a multi-subunit reverse transcriptase, or a variant or fragment thereof, and the sequence-specific nuclease, or a variant or active fragment thereof.

Also disclosed herein are nucleic acids encoding the disclosed polypeptide and expression vectors comprising the nucleic acid in combination with a promoter. Additionally disclosed are compositions comprising a polypeptide disclosed herein and a buffer.

Further disclosed are systems for modifying a target nucleic acid. The systems comprise a polypeptide as disclosed herein, or a nucleic acid encoding thereof; and one or more RNA polynucleotides comprising a spacer sequence and an extension sequence comprising a primer binding sequence (PBS) and a reverse transcriptase template (RTT) sequence, or one or more nucleic acids encoding thereof. In some embodiments, the spacer sequence and the extension sequence are contained within a single RNA polynucleotide. In some embodiments, the system further comprises a nicking guide RNA, or a nucleic acid encoding thereof. In some embodiments, the system further comprises a target nucleic acid.

Additionally, methods for modifying a target nucleic acid are disclosed. The methods comprise contacting the target nucleic acid with: a polypeptide as disclosed herein and one or more RNA polynucleotides comprising a spacer sequence and an extension sequence comprising a primer binding sequence (PBS) and a reverse transcriptase template (RTT) sequence. In some embodiments, the spacer sequence and the extension sequence are contained within a single RNA polynucleotide.

In some embodiments, the RTT sequence encodes one or more nucleotides to modify the target nucleic acid. In some embodiments, the RTT sequence encodes one or more nucleotide substitutions, additions, or deletions in reference to the target nucleic acid sequence.

In some embodiments, the method further comprises contacting the target nucleic acid with a nicking guide RNA (ngRNA).

In some embodiments, the target nucleic acid is genomic DNA. In some embodiments, the target nucleic acid encodes a gene.

In some embodiments, the target nucleic acid encodes a disease-causing mutation. In some embodiments, the RTT sequence encodes one or more nucleotide substitutions, additions, or deletions to correct the disease-causing mutation.

In some embodiments, the RTT sequence encodes one or more nucleotide substitutions, additions, or deletions to confer a disease-causing mutation in the target nucleic acid.

In some embodiments, the target nucleic acid is in a cell. In some embodiments, the cell is a eukaryotic cell. In some embodiments, the cell is a human cell. In some embodiments, the cell is in vitro. In some embodiments, the cell is ex vivo. In some embodiments, the cell is in vivo.

In some embodiments, the contacting comprises introducing to the cell: the polypeptide, or a nucleic acid encoding thereof; the one or more RNA polynucleotides, or one or nucleic acids encoding thereof; and optionally, the ngRNA, or a nucleic acid encoding thereof.

In some embodiments, the introducing into the cell comprises administering to a subject.

Methods for treating a disease or disorder in a subject are also disclosed. The methods comprise administering a system, as disclosed herein, to the subject. In some embodiments, the disease or disorder is associated with a disease-causing mutation. In some embodiments, the RTT sequence encodes one or more nucleotide substitutions, additions, or deletions to correct the disease-causing mutation.

Other aspects and embodiments of the disclosure will be apparent in light of the following detailed description.

### BRIEF DESCRIPTION OF THE DRAWINGS

FIG. 1 is a graph of the comparison of the optimized full-length and truncated MMLV reverse transcriptase-based prime editors for installation of transition, transversion, insertion, and deletion edits at two genomic loci, HEK3 and FANCF. Transversions: T to A (HEK3) and A to T (FANCF); Transitions: T to C (HEK3), A to G (FANCF); Insertions: Ins A (HEK3 and FANCF); Deletions: del T (HEK3) and del A (FANCF). Average of n=3 technical replicates for each edit.

FIG. 2 is a graph of the comparison of wildtype α- and β-subunit AMV reverse transcriptase-based prime editors for installation of transition, transversion, insertion, and deletion edits at two genomic loci, as described in FIG. 1. Average of n=3 technical replicates for each edit.

FIG. 3 is a graph of the comparison of wildtype α- and β-Subunit RSV reverse transcriptase-based prime editors for installation of transition, transversion, insertion, and deletion edits at two genomic loci, as described in FIG. 1. Average of n=3 technical replicates for each edit.

FIG. 4 is a graph of the comparison of wildtype p51 and p66-Subunit HIV reverse transcriptase-based prime editors for installation of transition, transversion, insertion, and deletion edits at two genomic loci, as described in FIG. 1. Average of n=3 technical replicates for each edit

FIG. 5 is a summary comparison of the deletion edit (del A) at FANCF, as indicated, for each of the reverse transcriptase-based prime editors and subunits thereof, using data from FIGS. 1-4. On the right is a chart listing the reverse transcriptase-based prime editors and subunits thereof and their size in bp.

FIG. 6 is a graph showing that modifications to the HIV reverse transcriptase p66-subunit modify prime editing efficiency at the HEK3 locus. The D549A mutation led to a statistically significant increase (p-value=0.0112) in prime editing efficiency compared to the unmodified HIV reverse transcriptase p66-subunit-based prime editor.

### DETAILED DESCRIPTION

Currently, prime editors use a Cas9 nickase linked to an optimized murine leukemia virus (MLV) reverse-transcriptase (RT) to facilitate prime editing. These prime editors are too large for packaging in a single adeno-associated viral vector for delivery to cells, thus necessitating a multi-vector strategy for prime editing (e.g., a split-intein prime editor viral construct). The disclosed systems, compositions, and methods comprise a mini-DNA synthesizer having a single subunit of a reverse transcriptase to facilitate DNA synthesis from an RNA template combined with a sequence-specific nuclease (e.g., Cas9, TALENs or ZFNs). The mini-DNA synthesizer enables precise installation and correction of mutations in a target nucleic acid. Due to its reduced size, the mini-DNA synthesizer allows easy packaging in non-viral nanoparticles and viral vector(s) in addition to being used in RNA-protein complexes. Additionally, the mini-DNA synthesizer may facilitate use of a single viral vector (e.g., a single AAV vector) or allow a more optimal and/or efficient split-intein site when using a two vector system.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” and “introducing,” are used interchangeably herein and refer to the placement into a cell, organism, or subject by a method or route which results in at least partial localization to a desired site. Administration can be by any appropriate route which results in delivery to a desired location in the cell, organism, or subject.

The term “contacting” as used herein refers to bring or put in contact, to be in or come into contact. The term “contact” as used herein refers to a state or condition of touching or of immediate or local proximity.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full-length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

A cell has been “genetically modified,” “transformed,” or “transfected” by exogenous DNA, e.g., a recombinant expression vector, when such DNA has been introduced inside the cell. The presence of exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. Peptides and polypeptides include proteins such as binding proteins, receptors, and antibodies.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of devices and systems contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods herein, the mammal is a human.

As used herein, “treat,” “treating,” and the like means a slowing, stopping, or reversing of progression of a disease or disorder. The term also includes a reversing of the progression of such a disease or disorder to a point of eliminating or greatly reducing the disease. As such, “treating” means an application or administration where the purpose is to cure, heal, alleviate, relieve, alter, remedy, ameliorate, improve, or affect the disease or symptoms of the disease.

A “vector” or “expression vector” is a replicon, such as plasmid, phage, virus, or cosmid, to which another DNA segment, e.g., an “insert,” may be attached or incorporated so as to bring about the replication of the attached segment in a cell.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Prime Editing

Prime editing is a double-strand break (DSB)-independent clustered-regularly interspaced short palindromic repeats (CRISPR)/CRISPR-associated (Cas) system that can ameliorate both transition and transversion mutations in addition to small deletions and insertions. Generally, a prime editing guide RNA (pegRNA) is used in conjunction with a prime editor, e.g., a H840A Streptococcus pyogenes Cas9 (spCas9) nickase linked to an optimized Moloney murine leukemia virus (MMLV) reverse transcriptase (RT).

pegRNAs are similar to standard single-guide RNAs (sgRNAs) but differ due to a sequence comprising a primer binding site (PBS) and a reverse transcription template (RTT) sequence. The primer binding site hybridizes with the bases upstream of the prime editor generated nick, while the RTT encodes the information of the intended edits and directs reverse transcription. Together, the prime editor and the pegRNA form the prime editing 2 strategy (PE2). The Cas9 nickase is guided to the DNA target site by the pegRNA. After nicking by Cas9, the reverse transcriptase uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Once the prime editor incorporates the edit into one strand, there is a mismatch between the original sequence on one strand and the edited sequence on the other strand. In some embodiments, an additional nicking guide RNA (ngRNA) is used to nick the non-edited strand, directing DNA repair enzymes to use the edited strand as a template to remake the mismatched strand. The prime editor, the pegRNA, and ngRNA form prime editing 3 (PE3) strategies.

### Mini-DNA Synthesizer

Disclosed herein is a polypeptide of a mini-DNA synthesizer comprising a single subunit of a multi-subunit reverse transcriptase and a sequence-specific nuclease for use in prime-editing. Using a single subunit of a multi-subunit reverse transcriptase, or a variant or fragment thereof provides advantages over a single-subunit reverse transcriptases due to its smaller size. For example, a single subunit of a multi-subunit reverse transcriptase, or a variant or fragment thereof, suitable for use herein are smaller (e.g., are encoded by a nucleic acid substantially shorter) than similar single-subunit reverse transcriptases (e.g., M-MLV reverse transcriptase). In some embodiments, the single subunit of a multi-subunit reverse transcriptase, or a variant or fragment thereof comprises less than 800 amino acids. In some embodiments, the single subunit of a multi-subunit reverse transcriptase, or a variant or fragment thereof comprises less than 700 amino acids. In some embodiments, the single subunit of a multi-subunit reverse transcriptase, or a variant or fragment thereof, comprises less than 600 amino acids. The single subunit of a multi-subunit reverse transcriptase, or a variant or fragment thereof, may be between 400 and 1000 amino acids. In some embodiments, the single subunit of a multi-subunit reverse transcriptase, or a variant or fragment thereof, is 400 to 1000 amino acids, 500 to 1000 amino acids, 600 to 1000 amino acids, 700 to 1000 amino acids, 800 to 1000 amino acids, 900 to 1000 amino acids, 400 to 900 amino acids, 500 to 900 amino acids, 600 to 900 amino acids, 700 to 900 amino acids, 800 to 900 amino acids, 400 to 800 amino acids, 500 to 800 amino acids, 600 to 800 amino acids, 700 to 800 amino acids, 400 to 700 amino acids, 500 to 700 amino acids, 600 to 700 amino acids, 400 to 600 amino acids, 500 to 600 amino acids, or 400 to 500 amino acids.

Reverse transcriptases, also known as RNA-dependent DNA polymerases, synthesize complementary DNA using RNA as a template. As used herein, the term “reverse transcriptase” encompasses any enzyme which synthesizes a complementary DNA using an RNA template.

RNA-dependent DNA polymerase activity and RNase activity are predominant functions of many reverse transcriptases. RNA-dependent DNA polymerase activity synthesizes the complementary DNA strand, incorporating dNTPs, whereas RNase activity degrades the RNA template of the DNA:RNA complex.

In some embodiments, the single subunit of a multi-subunit reverse transcriptase, or a variant or fragment thereof comprises a ribonuclease (RNase) domain (e.g., an RNaseH domain) in addition to the RNA-dependent DNA polymerase domain. In some embodiments, the single subunit of a multi-subunit reverse transcriptase, or a variant or fragment thereof comprises a truncated RNaseH domain. In some embodiments, the single subunit of a multi-subunit reverse transcriptase, or a variant or fragment thereof, lacks an RNaseH domain.

In some embodiments, the single subunit of a multi-subunit reverse transcriptase, or a variant or fragment thereof comprises a mutated RNaseH domain. In some embodiments, the mutations increase the stability or activity of the reverse transcriptase. In some embodiments, the mutations partially or fully abolish RNase H activity. See, for example, Konishi, A., et al, Biotechnology letters (2012), 34(7): 1209-1215, incorporate herein by reference in its entirety. The single subunit of a multi-subunit reverse transcriptase, or a variant or fragment thereof, may comprise mutations in the polymerization domain. For example, mutation in the polymerization domain may increase RNA-dependent DNA polymerase activity (e.g., processivity, efficiency, rate of incorporation of nucleotides).

In some embodiments, the single subunit of a multi-subunit reverse transcriptase, or a variant or fragment thereof comprises a connection subdomain, which connects the polymerase domain with the RNaseH domain. In some embodiments, the single subunit of a multi-subunit reverse transcriptase, or a variant or fragment thereof lacks a connection subdomain.

The disclosed polypeptides are not limited by the source of the single subunit of a multi-subunit reverse transcriptase. Reverse transcriptases may be from retroviruses, dsRNA viruses, and various retroelements in eukaryotes and prokaryotes.

In some embodiments, the single subunit of a multi-subunit reverse transcriptase, or a variant or fragment thereof is derived from a viral reverse transcriptase. In some embodiments, the single subunit of a multi-subunit reverse transcriptase, or a variant or fragment thereof is derived from: avian myeloblastosis virus (AMV) reverse transcriptase, Rous sarcoma virus (RSV) transcriptase, or HIV-1. In some embodiments, the single subunit of a multi-subunit reverse transcriptase, or a variant or fragment thereof is derived from: avian myeloblastosis virus reverse transcriptase (AMV RT)-alpha subunit, Rous sarcoma virus transcriptase (RSV RT)-alpha subunit, or HIV-1 reverse transcriptase (RT) p66 subunit.

In some embodiments, the single subunit of a multi-subunit reverse transcriptase, or a variant or fragment thereof comprises an amino acid sequence having at least 70% identity (e.g., at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity) to any of SEQ ID NOs: 4, 8, 9, 14, or 16.

Any of the single subunits of a multi-subunit reverse transcriptase, or a variant or fragment described herein may comprise one or more (e.g., 1, 2, 3, 4, 5, 6, 7, 8, 9, 10, 15, 20, 25, 50, 100, 150, 200, etc.) amino acid substitutions. As described elsewhere, the mutations may increase the stability or activity of the reverse transcriptase, partially or fully abolish RNase H activity, or a combination thereof.

An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

For example, one or more mutations may be incorporated into any of SEQ ID NOs: 4, 8, 9, 14, or 16 which increase editing efficiency.

As described herein, one or more mutations may be introduced into SEQ ID NO: 9. In some embodiments, the single subunit of a multi-subunit reverse transcriptase, or a variant or fragment thereof comprises an amino acid sequence having at least 70% identity of SEQ ID NO: 9 and one or more mutations at positions: D450, E484, and D505. In select embodiments, the single subunit of a multi-subunit reverse transcriptase, or a variant or fragment thereof comprises an amino acid sequence having at least 70% identity of SEQ ID NO: 9 and one or more mutations selected from: D450A, E484A, and D505A.

One or more mutations may be introduced into SEQ ID NO: 16. In some embodiments, the single subunit of a multi-subunit reverse transcriptase, or a variant or fragment thereof comprises an amino acid sequence having at least 70% identity of SEQ ID NO: 16 and one or more mutations at positions: L234, W402, W406, D443, E478, D498, and D549. In select embodiments, the single subunit of a multi-subunit reverse transcriptase, or a variant or fragment thereof comprises an amino acid sequence having at least 70% identity of SEQ ID NO: 16 and one or more mutations selected from: L234, W402, W406, D443, E478, D498, and D549.

Exemplary sequence-specific nucleases for use in the mini-DNA synthesizer include, but are not limited to, Cas proteins, Argonaute (Ago) proteins, zinc finger nucleases (ZFNs), and transcription activator-like effector nucleases (TALEN). In some embodiments, the sequence-specific nuclease is a Cas protein.

Cas proteins are described in further detail in, e.g., Haft et al., PLoS Comput. Biol., 1(6): e60 (2005), incorporated herein by reference. The Cas protein may be any Cas endonuclease, or fragment or naturally-occurring or engineered variants thereof. In some embodiments, the Cas endonuclease is a Class 2 Cas endonuclease. In some embodiments, the Cas endonuclease is a Type V Cas endonuclease. In some embodiments, the Cas protein is Cas9, Cas12a, otherwise referred to as Cpf1, or Cas14. In one embodiment, the Cas9 protein is a wild-type Cas9 protein. In some embodiments, the Cas9 protein is a Cas9 variant.

The Cas9 protein can be obtained or derived from any suitable microorganism, and a number of bacteria express Cas9 protein orthologs or variants. In some embodiments, the Cas9 is from Streptococcus pyogenes or Staphylococcus aureus. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases.

In certain embodiments, a Cas nuclease can only cleave a target sequence if an appropriate PAM is present. See, for example Doudna et al., Science, 2014, 346(6213): 1258096, incorporated herein by reference. A PAM site is a nucleotide sequence in proximity to a target sequence. For example, PAM site may be a DNA sequence immediately following the DNA sequence targeted by the Cas protein. A PAM can be 5′ or 3′ of a target sequence. A PAM can be upstream or downstream of a target sequence. A PAM can be 1, 2, 3, 4, 5, 6, 7, 8, 9, 10 or more nucleotides in length. In certain embodiments, a PAM is between 2-6 nucleotides in length. Nonlimiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, and NGGNG, where “N” is any nucleotide.

In some embodiments, the Cas protein comprises a Cas variant configured to target an expanded or altered range of PAM sequences which may facilitate essentially PAMless cleavage. In some embodiments, the Cas protein comprises a variant of the Streptococcus pyogenes Cas9 enzyme selected from xCas9, Cas9-VQR, SpG and SpRY. See, for example, Walton et al., Science. 2020 Apr. 17; 368(6488):290-296, Hu, et al., Nature 2018; 556 (57-63), Kleinstiver et al., Nature 2015; 523(7561):481-5, Hu et al., Mol Plant 2016; 9, 43-945, incorporated herein by reference in their entirety.

The Cas protein may be fully or partially catalytically inactive. In some embodiments, the Cas protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known (see, e.g., U.S. Patent Application Publication 2017/0051312, incorporated herein by reference) and include, for example, Streptococcus pyogenes with point mutations at D10 or H840.

In some embodiments, the Cas protein is a catalytically inactive Cas9 (dCas9). A catalytically inactive Cas9 protein is typically engineered through the introduction of inactivating point mutations in both of the catalytic nuclease domains. Methods for generating catalytically inactive Cas9 include, for example, Streptococcus pyogenes with point mutations at D10 and H840.

The single subunit of a multi-subunit reverse transcriptase and the sequence specific nuclease may be linked in any orientation. In some embodiments, the N-terminus of the sequence specific nuclease is linked to the C-terminus of the single subunit of a multi-subunit reverse transcriptase. In some embodiments, the C-terminus of the sequence specific nuclease is linked to the N-terminus of the single subunit of a multi-subunit reverse transcriptase. In some embodiments, the N-terminus of the sequence specific nuclease is linked to the N-terminus of the single subunit of a multi-subunit reverse transcriptase. In some embodiments, the C-terminus of the sequence specific nuclease is linked to the C-terminus of the single subunit of a multi-subunit reverse transcriptase.

The polypeptide may further comprise a linker polypeptide between the single subunit of a multi-subunit reverse transcriptase and the sequence specific nuclease. The linker polypeptide may have any of a variety of amino acid sequences and be a variety of lengths (e.g., 4-100 amino acids). These linkers can be produced by using synthetic, linker-encoding oligonucleotides to couple the portions of the polypeptide or can be encoded by a nucleic acid sequence encoding the polypeptide. In some embodiments, the linker polypeptide is considered a flexible linker, facilitating some degree of orientation freedom for the multi-subunit reverse transcriptase and the sequence specific nuclease from each other. A variety of different linkers are considered suitable for use, including but not limited to, glycine-serine polymers, glycine-alanine polymers, and alanine-serine polymers.

The polypeptide may further comprise a nuclear localization signal (NLS). The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine. The NLS(s) may be at the N-terminus, the C-terminus, or a combination thereof of the single subunit of a multi-subunit reverse transcriptase and/or the sequence specific nuclease.

In some embodiments, the NLS is a monopartite sequence. A monopartite NLS comprises a single cluster of positively charged or basic amino acids. In some embodiments, the monopartite NLS comprises a sequence of K—K/R—X—K/R, wherein X can be any amino acid. Exemplary monopartite NLS sequences include those from the SV40 large T-antigen, c-Myc, and TUS-proteins.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 41), and the NLS of EGL-13, MSRRRKANPTKLSENAKKLAKEVEN (SEQ ID NO: 42). In some embodiments, the NLS comprises a bipartite SV40 NLS. In certain embodiments, the NLS comprises an amino acid sequence having at least 70% similarity to KRTADGSEFESPKKKRKV (SEQ ID NO: 43). In select embodiments, the NLS consists of an amino acid sequence of KRTADGSEFESPKKKRKV (SEQ ID NO: 43).

The polypeptide may further comprise an epitope tag (e.g., 3×FLAG tag, an HA tag, a Myc tag, and the like). In some embodiments, the epitope tag may be adjacent, either upstream or downstream, to a nuclear localization sequence. The epitope tag(s) may be at the N-terminus, a C-terminus, or a combination thereof of the single subunit of a multi-subunit reverse transcriptase and/or the sequence specific nuclease.

### Systems

Disclosed herein are systems for modifying a target nucleic acid. The methods and systems comprise a polypeptide of a mini-DNA synthesizer as described herein, or a nucleic acid encoding thereof; and one or more RNA polynucleotides comprising a spacer sequence and an extension sequence comprising a primer binding sequence (PBS) and a reverse transcriptase template (RTT) sequence, or one or more nucleic acids encoding thereof. In some embodiments, the systems and methods further comprise a nicking guide RNA (ngRNA), or a nucleic acid encoding thereof.

1. pegRNA

The systems include a spacer sequence and an extension sequence comprising a primer binding sequence (PBS) and a reverse transcriptase template (RTT) sequence, or one or more nucleic acids encoding thereof. In some embodiments, each of the spacer sequence, PBS, and RTT sequence are provided as a single prime editing guide RNA (pegRNA), or a nucleic acid encoding thereof. The spacer sequence directs the nuclease to bind to a DNA molecule having complementarity with the pegRNA, the PBS hybridizes with the bases upstream of the nuclease generated nick, and the RTT encodes the information of the intended edits and directs reverse transcription.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization.

The pegRNAs may comprise additional structural elements or sequences including a gRNA scaffold responsible for binding to the sequence-specific nuclease, a transcription termination sequence that the 3′ end of the molecule, and mutations or structural motifs that increase editing efficiency or enhance RNA stability or prevent RNA degradation. For example, the pegRNA may further comprise: a triple helix forming sequence (e.g., triple helix terminators from a long non-coding RNAs (lncRNAs), e.g., metastasis-associated lung adenocarcinoma transcript 1 (MALAT1)); a tRNA-like sequence; a pseudoknot (e.g., a modified prequeosine₁-1 riboswitch aptamer, (evopreQ₁) or the frameshifting pseudoknot from Moloney murine leukemia virus (MMLV)); and silent mutations near the intended edit (e.g., less than 10 bp away). See, for example, Nelson, et al. Nat Biotechnol. 2022 March; 40(3):402-410, Chen, et al., Cell. 2021 Oct. 28; 184(22):5635-5652.e29, International Patent Publication No. WO2022067130, each of which is incorporated herein by reference in its entirety.

The additional structural elements or sequences may be present at any location in the pegRNA which does not interfere with the function of the spacer sequence, primer binding sequence (PBS), and a reverse transcriptase template (RTT) sequence. In some embodiments, the additional structural elements or sequences are at the 3′ end of the pegRNA.

2. Nicking Guide RNA (ngRNA)

In some embodiments, the systems and methods further comprise a nicking guide RNA (ngRNA) that complexes with the sequence-specific nuclease and introduces a nick in the non-edited DNA stand, or a nucleic acid encoding thereof. In certain embodiments, the nick induced by using the ngRNA is on the opposite strand as the initial nick. In certain embodiments, the nick induced by using the ngRNA is on the same strand as the initial nick. Thus, the ngRNA sequence may target the same or different strand as the spacer sequence. In some embodiments, the ngRNA may improve the efficiency of the system.

### Nucleic Acids

The present disclosure also provides for one or more nucleic acids encoding the mini-DNA synthesizer, pegRNA, and ngRNA, disclosed herein, vectors containing these nucleic acids and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

In some embodiments, the one or more nucleic acids comprise one or more messenger RNAs, one or more vectors, or any combination thereof. In some embodiments, the one or more nucleic acids includes a messenger RNA for expression of the mini-DNA synthesizer and at least one nucleic acid provides the pegRNA and ngRNA. A single nucleic acid may encode the mini-DNA synthesizer and the pegRNA and ngRNA, or the mini-DNA synthesizer can be encoded on a separate nucleic acid from the pegRNA and ngRNA.

In some embodiments, the mini-DNA synthesizer is provided as a split-enzyme such that two separate proteins together form a functional mini-DNA synthesizer. In some such cases the sequences that encode the two parts of the split-protein are present on the same vector. In some cases, they are present on separate vectors, e.g., as part of a vector system that encodes the mini-DNA synthesizer, pegRNA, ngRNA and systems thereof. Split systems include, but are not limited to, intein, MS2, or SunTag based systems. The split system may comprise more than one split system type (e.g., an intein based system and a SunTag based system) or more than one split system of a single type (e.g., one or more intein based systems).

Nucleic acids of the present disclosure (e.g., nucleic acids encoding the polypeptide of a mini-DNA synthesizer described herein, pegRNA, ngRNA, nucleic acids encoding pegRNA and ngRNA) can comprise any of a number of promoters, including, but not limited to, constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence that is capable of driving expression of the desired protein operably linked thereto.

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene for selection of stable or transient transfectants in host cells; transcription termination and RNA processing signals; 5′- and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA.

Thus, the disclosure further provides for cells comprising a system for modifying a target nucleic acid, or one or more nucleic acids or vectors encoding thereof, as disclosed herein.

Conventional viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells, tissues and/or a subject. Viral vectors include, for example, retroviral, lentiviral, adenoviral, adeno-associated, baculoviral, and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant baculoviruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell.

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

As such, the disclosure provides an isolated cell comprising the vector(s) or nucleic acid(s) disclosed herein. Preferred cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993), incorporated herein by reference. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines. Methods for selecting suitable mammalian cells and methods for transformation, culture, amplification, screening, and purification of cells are known in the art.

In some embodiments, the cell is a eukaryotic cell. In some embodiments, the cell is a mammalian cell. In some embodiments, the cell is a human cell. In some embodiments, the cell is in vitro. In some embodiments, the cell is ex vivo. In some embodiments, the cell is in vivo and delivery to the cell comprises administration to a subject.

### Methods

The disclosure also provides methods of altering a target nucleic acid sequence (e.g., DNA or RNA). The phrase “altering a nucleic acid sequence,” as used herein, refers to modifying at least one physical feature of a nucleic acid sequence of interest. Nucleic acid alterations include, for example, single or double strand breaks, deletion, or insertion of one or more nucleotides, and other modifications that affect the structural integrity or nucleotide sequence of the nucleic acid sequence.

The methods comprise contacting a target nucleic acid sequence with the polypeptide described herein, or a nucleic acid encoding thereof; one or more RNA polynucleotides comprising a spacer sequence and an extension sequence comprising a primer binding sequence (PBS) and a reverse transcriptase template (RTT) sequence, or one or more nucleic acids encoding thereof; and optionally, a nicking guide RNA (ngRNA), or a nucleic acid encoding thereof. In some embodiments, the methods comprise contacting a target nucleic acid sequence with a system as described herein.

In some embodiments, the target nucleic acid is in a cell. Suitable cells include, but are not limited to: bacterial cell; an archaeal cell; a eukaryotic cell; a cell of a single-cell eukaryotic organism; a plant cell; a protozoa cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell of a vertebrate animal (e.g., a fish, an amphibian, a reptile, a bird, a mammal); a cell of a mammal (e.g., a cell of a rodent; a cell of a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuna, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). In some cases, the cell is a cell that does not originate from a natural organism (e.g., the cell can be a synthetically made cell; also referred to as an artificial cell).

In some embodiments, the cell is a eukaryotic cell. In some embodiments, the cell is a human cell. In some embodiments, the cell is in vitro. In some embodiments, the cell is ex vivo. In some embodiments the cell is in vivo.

In some embodiments, the target nucleic acid is a nucleic acid endogenous to a target cell. In some embodiments, the target nucleic acid is genomic DNA. In some embodiments, the target nucleic acid encodes a gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

In some embodiments, the RTT sequence encodes one or more nucleotides to modify the target nucleic acid.

In some embodiments, contacting the target nucleic acid comprises introducing into the cell: a polypeptide as described herein, or a nucleic acid encoding thereof; one or more RNA polynucleotides comprising a spacer sequence and an extension sequence comprising a primer binding sequence (PBS) and a reverse transcriptase template (RTT) sequence, or one or more nucleic acids encoding thereof; and optionally, a nicking guide RNA (ngRNA), or a nucleic acid encoding thereof. In some embodiments, the methods comprise introducing into the cell a system as described herein.

In some embodiments, introducing into the cell comprises administering to the subject. The methods comprise administering to a subject: a polypeptide as described herein, or a nucleic acid encoding thereof; and one or more RNA polynucleotides comprising a spacer sequence and an extension sequence comprising a primer binding sequence (PBS) and a reverse transcriptase template (RTT) sequence, or one or more nucleic acids encoding thereof; and optionally, a nicking guide RNA (ngRNA), or a nucleic acid encoding thereof. The systems and components disclosed herein are applicable to the methods for treatment and prevention of a disease or disorder.

In some embodiments, the systems and methods described herein may be used to correct one or more defects or mutations in a gene (referred to as “gene correction”). In such cases, the target sequence encodes a defective version of a gene, and the disclosed compositions and systems further comprise a nucleic acid molecule which encodes a wild-type or corrected version of the gene. The disclosed compositions and systems may be used to correct one or more mutations or defect in a single gene. The disclosed compositions and systems may be used to correct one or more mutations or defect in multiple genes.

The methods described here also provide for treating a disease or condition in a subject. The method may comprise administering to the subject, in vivo, or by transplantation of ex vivo treated cells, a therapeutically effective amount of a polypeptide as described herein, or a nucleic acid encoding thereof; and one or more RNA polynucleotides comprising a spacer sequence and an extension sequence comprising a primer binding sequence (PBS) and a reverse transcriptase template (RTT) sequence, or one or more nucleic acids encoding thereof; and optionally, a nicking guide RNA (ngRNA), or a nucleic acid encoding thereof. In some embodiments, the methods comprise administering to the subject, in vivo, or by transplantation of ex vivo treated cells, a therapeutically effective amount of system as disclosed herein.

In some embodiments, the systems and methods are used to treat a pathogen or parasite on or in a subject by altering the pathogen or parasite. In some embodiments, the systems and methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, bestrophin-1 (Best1), cystic fibrosis transmembrane conductance regulator (CFTR), crumbs cell polarity complex component 1 (CRB1), β-hemoglobin (HBB), oculocutaneous albinism II (OCA2), Huntingtin (HTT), dystrophia myotonica-protein kinase (DMPK), EGF containing fibulin extracellular matrix protein 1 (EFEMP1), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), rhodopsin (RHO), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1):192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the target DNA sequence can comprise a cancer oncogene.

The present disclosure provides for gene editing methods that can ablate a disease-associated gene, which in turn can be used for in vivo gene therapy for patients. In some embodiments, the gene editing methods include donor nucleic acids comprising therapeutic genes.

In some embodiments, systems and methods described herein may be used to insert or confer one or more defects or mutations in a gene. In such cases, the target sequence encodes a wild-type or normal version of the gene, and the disclosed compositions and systems comprise a nucleic acid molecule which encodes one or more nucleotide substitutions, additions, or deletions for a disease-causing version of the gene. For example, the disclosed compositions and systems may be used to install mutations for disease modeling in cells and organisms. The disclosed compositions and systems may be used to install one or more mutations or defects into a single gene. The disclosed compositions and systems may be used to install one or more mutations or defects into multiple genes.

Administration may be through any suitable mode of administration, including but not limited to: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, topical, and inhalation. In some embodiments, the systems or components are delivered to the tissue(s) of interest. Such delivery may be either via a single dose, or multiple doses.

In some embodiments, an effective amount of the components of the systems, methods or compositions as described can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful modification of the target nucleic acid or gene is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

### EXAMPLES

The following are examples of the present invention and are not to be construed as limiting.

### Example 1

### Editing Efficiency

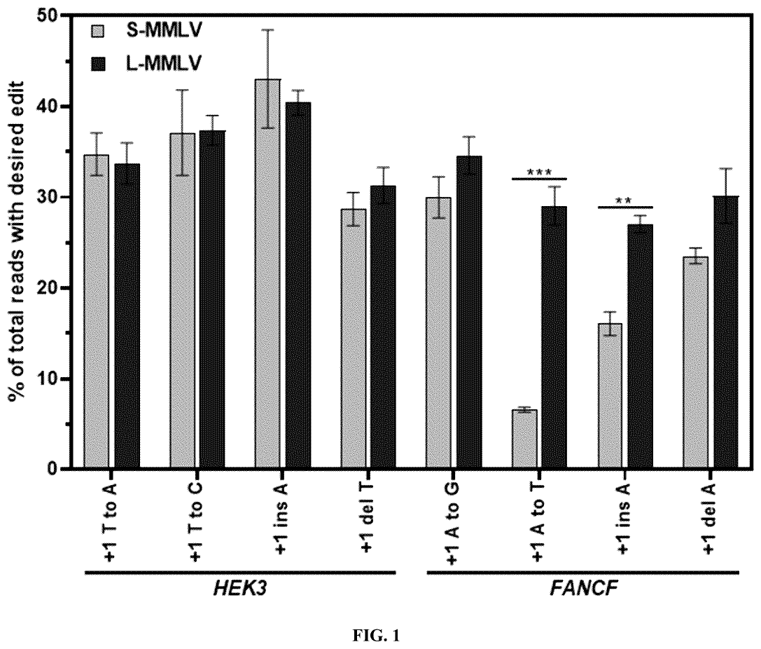

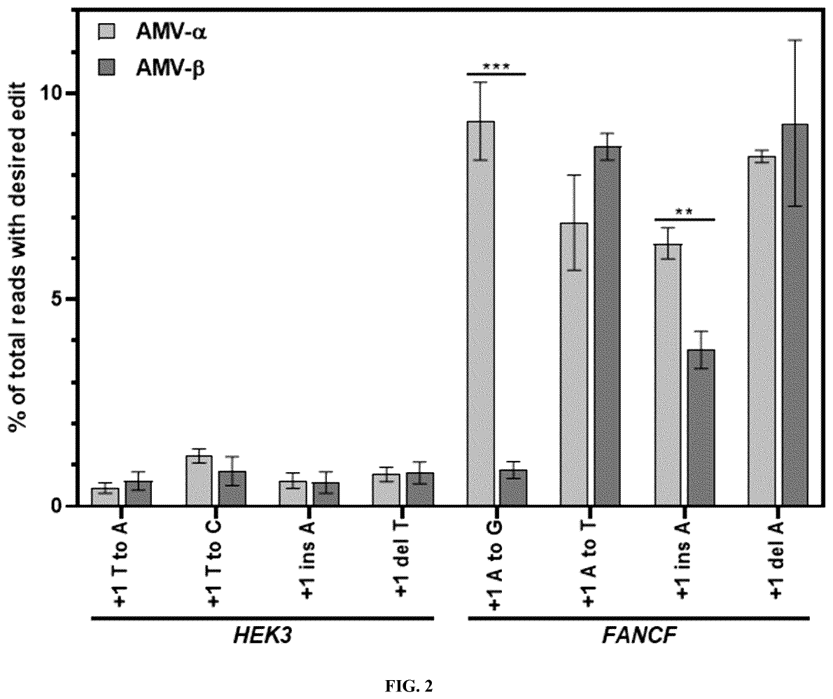

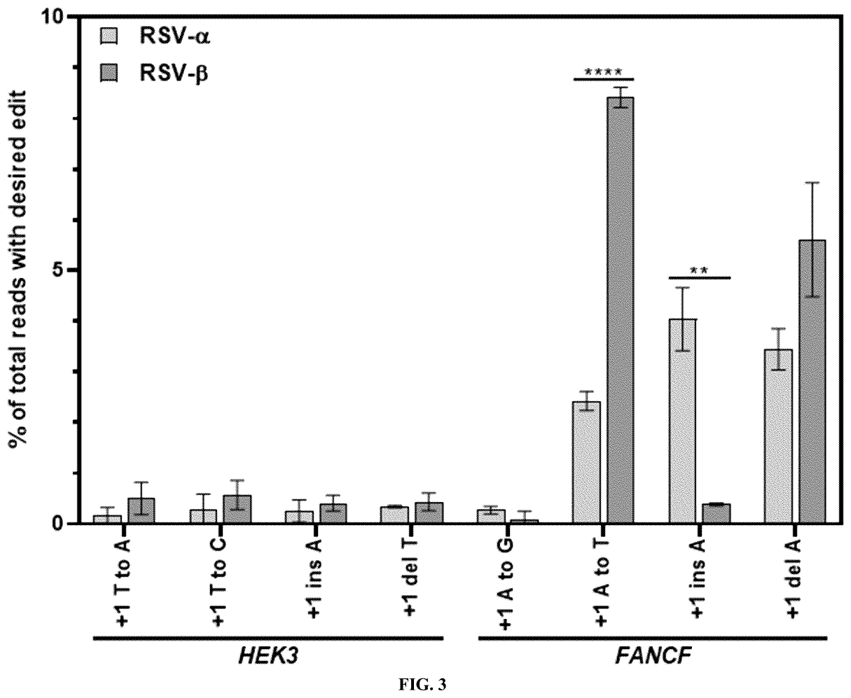

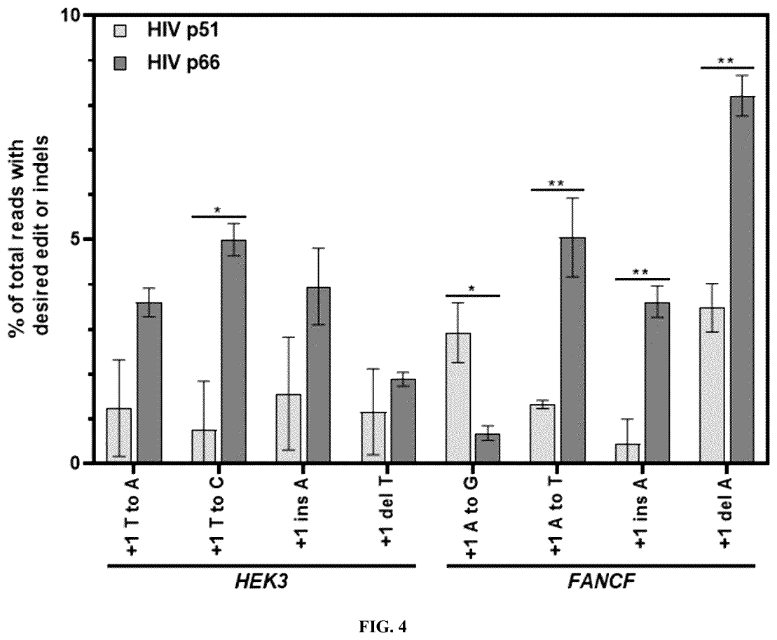

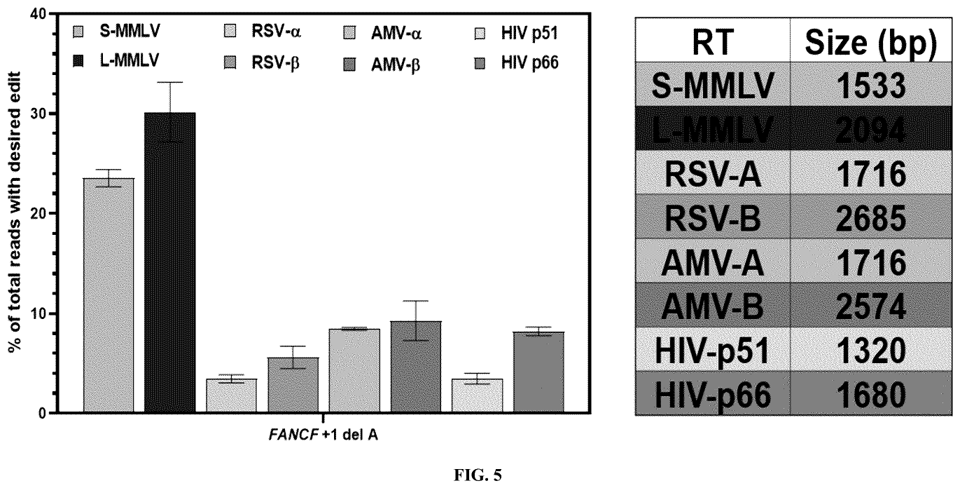

Reverse transcription variants, as indicated below, were tested by fusing to a SpCas9 to form a Cas9-mini DNA synthesizer fusion protein using a glycine/serine linker. Variant #4 is the full length MMLV reverse transcriptase without modifications. For all-in-one constructs (shown in the table below), the Cas9-mini DNA synthesizer variants were put into a plasmid together with U6 promoter-driven pegRNA and 7SK promoter-driven nicking gRNA. The pegRNA and nicking gRNA targets HEK3 locus and were designed insert a CTT triple nucleotide sequence. For separate constructs (FIGS. 1-6), the Cas9-mini DNA synthesizer variants, pegRNA (driven by U6 promoter) and nicking gRNA (driven by U6 promoter) were delivered as three separate plasmids. The pegRNA and nicking gRNA targets HEK3 or FANCF locus and were designed to install different types of edits, including transversions: T to A (HEK3) and A to T (FANCF); transitions: T to C (HEK3), A to G (FANCF); insertions: Ins A (HEK3 and FANCF); deletions: del T (HEK3) and del A (FANCF).

To test the editing efficiency, 1.5 ug of all-in-one plasmid (expressing Cas9-mini DNA synthesizer, pegRNA and gRNA) or separate plasmids (1050 ng of Cas9-mini DNA synthesizer, 393.75 ng of pegRNA and 78.75 ng of gRNA) were transfected into 5×10∝HEK293 cells in a 24-well plate setting using lipofectamine 2000. The lipofectamine and plasmid ratio is 1 microliter:1 microgram. After 72 hours of transfection, the dead cells were discarded and the cells were washed and collected. The genomic DNA of the cells were then extracted for PCR amplification of the target locus. The PCR amplicon of the target locus were analyzed by next-generation sequencing.

As shown below and in FIGS. 1-6, editing can be achieved using single subunits of a multi-subunit reverse transcriptase. For example, avian myeloblastosis virus reverse transcriptase (AMV RT)-alpha subunit showed 2-3 fold better editing efficiency avian myeloblastosis virus reverse transcriptase (AMV RT)-beta subunit, in either orientation. Thus, in some embodiments, the single subunit of a multi-subunit reverse transcriptase comprises the alpha subunit of the AMV RT and not the beta subunit of the AMV RT. The alpha subunit of Rous sarcoma virus RT and the p66 subunit of the HIV RT also enables successful editing. Introducing either a single mutation (D450A) or three mutations (D450A/E484A/D505A) into the AMV RT-alpha subunit (AMV-RT-a) led to better editing efficiencies compared to unedited AMV-RT-a, with the triple mutant showing the highest editing efficiency of the three variants. Editing was still measurable when removing some or all of the RNaseH domain. For example, removing the RNaseH domain and connecting subdomain, an N-terminal truncation of 258 amino acids, or removing a portion of the RNaseH domain, an N-terminal truncation of 123 amino acids, from AMV-RT-a still facilitated editing. Similarly, HIV p66-RT lacking the RNaseH domain enabled editing. Sequences of each of the variants in the following table are shown below.

| Variant | | | Editing |
|---|---|---|---|
| # | Variant Description | Size (bp) | Efficiency |
| V4 | MMLV-RT | 2094 bp | 60.90% |
| V7 | AMV-RT-ab | 4356 bp | 14.30% |
| V8 | AMV-RT-ba | 4356 bp | 7.60% |
| V9 | AMV-RT-a | 1716 bp | 7.00% |
| V10 | AMV-RT-b | 2574 bp | 2.40% |
| V11 | N-ter AMV-RT-a | 1716 bp | 5.10% |
| V12 | N-ter AMV-RT-b | 2574 bp | 3.40% |
| V13 | AMV-RT-a Single mutation on RNaseH | 1716 bp | 8.50% |
| V14 | AMV-RT-a Triple mutations on RNaseH | 1716 bp | 12.70% |
| V15 | AMV-RT, minus RNaseH and connection | 944 bp | 0.10% |
| V17 | AMV-RT, truncated RNaseH | 1349 bp | 0.10% |
| V19 | RSV-a RT | 1716 bp | 2.00% |
| V20 | Marathon RT | 1281 bp | 11.00% |
| V21 | HIV p66-RT | 1680 bp | 10.80% |
| V22 | HIV p66-RT minus RNaseH | 1317 bp | 2.50% |

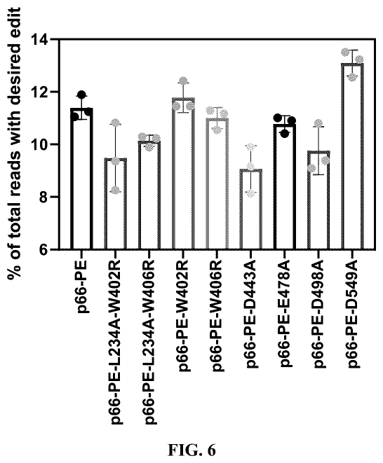

The WT HIV RT p66-subunit (V21) was modified by selectively mutating a number of different residues. The D549A mutation led to a statistically significant increase in editing efficiency (FIG. 6). Similar to the AMV-RT-a data editing efficiency of the single subunit RTs can be increased through mutations.

Sequences

| Variant # | Amino Acid Sequence | DNA Sequence |
|---|---|---|
| V4 | TLNIEDEYRLHETSKEPDVSLGSTWLSDFP | accctaaatatagaagatgagtatcggcta |
| | QAWAETGGMGLAVRQAPLIIPLKATSTPVS | catgagacctcaaaagagccagatgtttct |
| | IKQYPMSQEARLGIKPHIQRLLDQGILVPC | ctagggtccacatggctgtctgattttcct |
| | QSPWNTPLLPVKKPGTNDYRPVQDLREVNK | caggcctgggcggaaaccgggggcatggga |
| | RVEDIHPTVPNPYNLLSGLPPSHQWYTVLD | ctggcagttcgccaagctcctctgatcata |
| | LKDAFFCLRLHPTSQPLFAFEWRDPEMGIS | cctctgaaagcaacctctacccccgtgtcc |
| | GQLTWTRLPQGFKNSPTLFNEALHRDLADF | ataaaacaataccccatgtcacaagaagcc |
| | RIQHPDLILLQYVDDLLLAATSELDCQQGT | agactggggatcaagccccacatacagaga |
| | RALLQTLGNLGYRASAKKAQICQKQVKYLG | ctgttggaccagggaatactggtaccctgc |
| | YLLKEGQRWLTEARKETVMGQPTPKTPRQL | cagtccccctggaacacgcccctgctaccc |
| | REFLGKAGFCRLFIPGFAEMAAPLYPLTKP | gttaagaaaccagggactaatgattatagg |
| | GTLFNWGPDQQKAYQEIKQALLTAPALGLP | cctgtccaggatctgagagaagtcaacaag |
| | DLTKPFELFVDEKQGYAKGVLTQKLGPWRR | cgggggaagacatccaccccaccgtgccca |
| | PVAYLSKKLDPVAAGWPPCLRMVAAIAVLT | acccttacaacctcttgagcgggctcccac |
| | KDAGKLTMGQPLVILAPHAVEALVKQPPDR | cgtcccaccagtggtacactgtgcttgatt |
| | WLSNARMTHYQALLLDTDRVQFGPVVALNP | taaaggatgcctttttctgcctgagactcc |
| | ATLLPLPEEGLQHNCLDILAEAHGTRPDLT | accccaccagtcagcctctcttcgcctttg |
| | DQPLPDADHTWYTDGSSLLQEGQRKAGAAV | agtggagagatccagagatgggaatctcag |
| | TTETEVIWAKALPAGTSAQRAELIALTQAL | gacaattgacctggaccagactcccacagg |
| | KMAEGKKLNVYTDSRYAFATAHIHGEIYRR | gtttcaaaaacagtcccaccctgtttaatg |
| | RGWLTSEGKEIKNKDEILALLKALFLPKRL | aggcactgcacagagacctagcagacttcc |
| | SIIHCPGHQKGHSAEARGNRMADQAARKAA | ggatccagcacccagacttgatcctgctac |
| | ITETPDTSTLLIENSSPSGGSKRTADGSEF | agtacgtggatgacttactgctggccgcca |
| | E | cttctgagctagactgccaacaaggtactc |
| | (SEQ ID NO: 1) | gggccctgttacaaaccctagggaacctcg |
| | | ggtatcgggcctcggccaagaaagcccaaa |
| | | tttgccagaaacaggtcaagtatctggggt |
| | | atcttctaaaagagggtcagagatggctga |
| | | ctgaggccagaaaagagactgtgatggggc |
| | | agcctactccgaagacccctcgacaactaa |
| | | gggagttcctagggaaggcaggcttctgtc |
| | | gcctcttcatccctgggtttgcagaaatgg |
| | | cagcccccctgtaccctctcaccaaaccgg |
| | | ggactctgtttaattggggcccagaccaac |
| | | aaaaggcctatcaagaaatcaagcaagctc |
| | | ttctaactgccccagccctggggttgccag |
| | | atttgactaagccctttgaactctttgtcg |
| | | acgagaagcagggctacgccaaaggtgtcc |
| | | taacgcaaaaactgggaccttggcgtcggc |
| | | cggtggcctacctgtccaaaaagctagacc |
| | | cagtagcagctgggtggcccccttgcctac |
| | | ggatggtagcagccattgccgtactgacaa |
| | | aggatgcaggcaagctaaccatgggacagc |
| | | cactagtcattctggccccccatgcagtag |
| | | aggcactagtcaaacaaccccccgaccgct |
| | | ggctttccaacgcccggatgactcactatc |
| | | aggccttgcttttggacacggaccgggtcc |
| | | agttcggaccggtggtagccctgaacccgg |
| | | ctacgctgctcccactgcctgaggaagggc |
| | | tgcaacacaactgccttgatatcctggccg |
| | | aagcccacggaacccgacccgacctaacgg |
| | | accagccgctcccagacgccgaccacacct |
| | | ggtacacggatggaagcagtctcttacaag |
| | | agggacagcgtaaggcgggagctgcggtga |
| | | ccaccgagaccgaggtaatctgggctaaag |
| | | ccctgccagccgggacatccgctcagcggg |
| | | ctgaactgatagcactcacccaggccctaa |
| | | agatggcagaaggtaagaagctaaatgttt |
| | | atactgatagccgttatgcttttgctactg |
| | | cccatatccatggagaaatatacagaaggc |
| | | gtgggtggctcacatcagaaggcaaagaga |
| | | tcaaaaataaagacgagatcttggccctac |
| | | taaaagccctctttctgcccaaaagactta |
| | | gcataatccattgtccaggacatcaaaagg |
| | | gacacagcgccgaggctagaggcaaccgga |
| | | tggctgaccaagcggcccgaaaggcagcca |
| | | tcacagagactccagacacctctaccctcc |
| | | tcatagaaaattcatcaccctctggcggct |
| | | caaaaagaaccgccgacggcagcgaattcg |
| | | agcccaagaagaagaggaaagtc |
| | | (SEQ ID NO: 21) |
| V7 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLFSTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFTD | TTCCAATGGAAAGTCCTGCCTCAGGGTATG |
| | ASSSTHKGVVVWREGPRWEIKEIADLGASV | ACTTGCAGCCCAACAATTTGCCAGCTTATT |
| | QQLEARAVAMALLLWPTTPTNVVTDSAFVA | GTGGGACAAATCCTTGAACCCTTGAGACTT |
| | KMLLKMGQEGVPSTAAAFILEDALSQRSAM | AAACACCCAAGTCTCAGGATGCTCCACTAC |
| | AAVLHVRSHSEVPGFFTEGNDVADSQATFQ | ATGGACGACCTGCTTCTTGCTGCATCCAGC |
| | AYGSGATNFSLLKQAGDVEENPGPTVALHL | CATGATGGACTTGAGGCCGCCGGCGAAGAA |
| | AIPLKWKPNHTPVWIDQWPLPEGKLVALTQ | GTTATATCAACTTTGGAACGCGCTGGATTC |
| | LVEKELQLGHIEPSLSCWNTPVFVIRKASG | ACTATATCACCAGATAAAGTACAGCGGGAG |
| | SYRLLHDLRAVNAKLVPFGAVQQGAPVLSA | CCTGGGGTGCAGTATCTGGGTTACAAGCTC |
| | LPRGWPLMVLDLKDCFFSIPLAEQDREAFA | GGCTCCACATATGTCGCCCCTGTCGGACTG |
| | FTLPSVNNQAPARRFQWKVLPQGMTCSPTI | GTGGCCGAGCCTAGAATCGCAACACTGTGG |
| | CQLIVGQILEPLRLKHPSLRMLHYMDDLLL | GACGTTCAAAAATTGGTAGGGTCACTGCAA |
| | AASSHDGLEAAGEEVISTLERAGFTISPDK | TGGTTGAGACCTGCCCTCGGCATTCCCCCC |
| | VQREPGVQYLGYKLGSTYVAPVGLVAEPRI | CGGTTGATGGGACCATTTTACGAACAATTG |
| | ATLWDVQKLVGSLQWLRPALGIPPRLMGPF | CGGGGAAGCGATCCAAACGAAGCACGAGAG |
| | YEQLRGSDPNEAREWNLDMKMAWREIVQLS | TGGAATCTGGATATGAAAATGGCCTGGCGC |
| | TTAALERWDPALPLEGAVARCEQGAIGVLG | GAAATTGTACAGCTGAGTACAACAGCAGCA |
| | QGLSTHPRPCLWLESTQPTKAFTAWLEVLT | TTGGAACGATGGGACCCCGCTCTGCCTCTT |
| | LLITKLRASAVRTFGKEVDILLLPACFRED | GAAGGAGCTGTGGCCCGATGCGAGCAGGGA |
| | LPLPEGILLALRGFAGKIRSSDTPSIFDIA | GCTATCGGTGTCCTTGGCCAAGGGTTGAGC |
| | RPLHVSLKVRVTDHPVPGPTVFTDASSSTH | ACTCACCCTCGACCATGTTTGTGGTTGTTT |
| | KGVVVWREGPRWEIKEIADLGASVQQLEAR | AGCACTCAGCCTACTAAAGCATTTACCGCC |
| | AVAMALLLWPTTPTNVVTDSAFVAKMLLKM | TGGCTCGAGGTCCTCACATTGTTGATAACC |
| | GQEGVPSTAAAFILEDALSQRSAMAAVLHV | AAACTTCGCGCAAGCGCAGTGCGGACCTTC |
| | RSHSEVPGFFTEGNDVADSQATFQAYPLRE | GGAAAAGAAGTCGACATTCTGTTGCTCCCT |
| | AKDLHTALHIGPRALSKACNISMQQAREVV | GCCTGTTTCAGGGAAGACCTTCCATTGCCC |
| | QTCPHCNSAPALEAGVNPRGLGPLQIWQTD | GAGGGCATATTGCTGGCCCTCAGAGGCTTC |
| | FTLEPRMAPRSWLAVTVDTASSAIVVTQHG | GCTGGAAAGATCCGAAGTTCAGACACCCCA |
| | RVTSVAAQHHWATAIAVLGRPKAIKTDNGS | TCCATCTTCGATATAGCACGCCCTCTGCAT |
| | CFTSKSTREWLARWGIAHTTGIPGNSQGQA | GTATCCCTTAAAGTGAGGGTAACTGACCAC |
| | MVERANRLLKDKIRVLAEGDGFMKRIPTSK | CCTGTACCCGGACCCACCGTCTTCACAGAT |
| | QGELLAKAMYALNHFERGENTKTPIQKHWR | GCTTCATCTTCAACCCACAAAGGCGTAGTG |
| | PTVLTEGPPVKIRIETGEWEKGWNVLVWGR | GTTTGGCGAGAGGGCCCCAGGTGGGAGATA |
| | GYAAVKNRDTDKVIWVPSRKVKPDIAQKDE | AAAGAAATAGCCGACCTCGGGGCAAGCGTT |
| | VTKKDEASPLFA | CAACAGCTTGAGGCCAGAGCAGTGGCTATG |
| | (SEQ ID NO: 2) | GCCTTGCTGCTGTGGCCTACTACTCCCACC |
| | | AATGTTGTGACAGATTCTGCCTTCGTCGCA |
| | | AAGATGCTGCTGAAGATGGGCCAAGAGGGC |
| | | GTTCCTTCAACCGCTGCTGCATTTATTCTC |
| | | GAGGATGCTCTTAGCCAAAGGTCCGCCATG |
| | | GCAGCCGTTCTTCACGTGCGGTCACATTCT |
| | | GAAGTGCCAGGATTCTTCACTGAGGGCAAT |
| | | GATGTCGCAGACTCOCAAGCAACCTTCCAG |
| | | GCATATGGCAGCGGCGCAACTAACTTTTCC |
| | | TTCCAGGCATATGGCAGCGGCGCAACTAAC |
| | | TTTTCCTTGTTGAAGCAGGCTGGCGATGTA |
| | | GAGGAGAATCCTGGTCCAactgttgcgcta |
| | | catctggctattccgctcaaatggaagcca |
| | | aaccacacgcctgtgtggattgaccagtgg |
| | | ccccttcctgaaggtaaacttgtagcgcta |
| | | acgcaattagtggaaaaagaattacagtta |
| | | ggacatatagaaccttcacttagttgctgg |
| | | aacacacctgtctttgtgatccggaaggct |
| | | tccgggtcttatcgcttattgcatgacttg |
| | | cgcgctgttaacgctaagcttgttcctttt |
| | | ggggccgtccaacagggggcgccggttctc |
| | | tccgcgctcccgcgtggctggcccctgatg |
| | | gtcctagacctcaaggattgcttcttttct |
| | | attcctcttgcggaacaagatcgcgaagct |
| | | tttgcatttacgctcccctctgtgaataac |
| | | caggcccccgctcgaagattccaatggaag |
| | | gtcttgccccaagggatgacctgttctccc |
| | | actatctgtcagttgatagtgggtcaaata |
| | | cttgagcccttgcgactcaagcacccatct |
| | | ctgcgcatgttgcattatatggatgatctt |
| | | ttgctagccgcctcaagtcatgatgggttg |
| | | gaagcggcaggggaggaggttatcagtaca |
| | | ttggaaagagccgggttcaccatttcgcct |
| | | gataaggtccagagggagcccggagtacaa |
| | | tatcttgggtacaagttaggcagtacgtat |
| | | gtagcacccgtaggcctggtagcagaaccc |
| | | aggatagccaccttgtgggatgttcagaag |
| | | ctggtggggtcacttcagtggcttcgccca |
| | | gcgctaggaatcccgcctcgactgatgggc |
| | | cccttttatgagcagttacgagggtcagat |
| | | cctaacgaggcgagggaatggaatctagac |
| | | atgaaaatggcctggagagagatcgtgcag |
| | | ctcagcaccactgctgccttggaacgatgg |
| | | gaccctgccctgcctctggaaggagcggtc |
| | | gctagatgtgaacagggggcaataggggtc |
| | | ctgggacagggactgtccacacacccaagg |
| | | ccatgtttgtggttattctccacccaaccc |
| | | accaaggcgtttactgcttggttagaagtg |
| | | ctcacccttttgattactaagctacgtgct |
| | | tcggcagtgcgaacctttggaaggaggttg |
| | | atatcctcctgttgcctgcatgctttcggg |
| | | aggaccttccgctcccggaggggatcctgt |
| | | tagcccttagggggtttgcaggaaaaatca |
| | | ggagtagtgacacgccatctatttttgaca |
| | | ttgcgcgtccactgcatgtttctctgaaag |
| | | tgagggttaccgaccaccctgtaccgggac |
| | | ccactgtctttaccgacgcctcctcaagca |
| | | cccataagggggtggtagtctggagggagg |
| | | gcccaaggtgggagataaaagaaatagctg |
| | | atttgggggcaagtgtacaacaactggaag |
| | | cacgcgctgtggccatggcacttctgctgt |
| | | ggccgacaacgcccactaatgtagtgactg |
| | | actctgcgtttgttgcgaaaatgttactca |
| | | agatggggcaggagggagtcccgtctacag |
| | | cggcggcttttattttagaggatgcgttaa |
| | | gccaaaggtcagccatggccgccgttctcc |
| | | acgtgcggagtcattctgaagtgccagggt |
| | | ttttcacagaaggaaatgacgtggcagata |
| | | gccaagccacctttcaagcgtatcccttga |
| | | gagaggctaaagatctccataccgctctcc |
| | | atatcggaccccgcgcgctatccaaagcgt |
| | | gtaatatatctatgcagcaggctagggagg |
| | | ttgttcagacctgcccgcattgtaattcag |
| | | cccctgcgttggaggccggggtaaacccta |
| | | ggggtttgggacccctacagatatggcaga |
| | | cagactttacactagagcctagaatggctc |
| | | cccgttcctggctcgctgttactgtggata |
| | | ccgcctcatctgcgatagtcgtaactcagc |
| | | atggccgtgtcacatcggttgctgcacaac |
| | | atcattgggccacggctatcgccgttttgg |
| | | gaagaccaaaggccataaaaacagataatg |
| | | ggtcctgcttcacgtctaaatccacgcgag |
| | | agtggctcgcgagatgggggatagcacaca |
| | | ccaccgggattccgggtaattcccagggtc |
| | | aagctatggtagagcgggccaaccggctcc |
| | | tgaaagataagatccgtgtgcttgcggagg |
| | | gggatggctttatgaaaagaatccccacca |
| | | gcaaacagggggaactattagccaaggcaa |
| | | tgtatgcccttaatcactttgagcgtggtg |
| | | aaaacacaaaaacaccgatacaaaaacact |
| | | ggagacctaccgttcttacagaaggacccc |
| | | cggttaaaatacgaatagagacaggggagt |
| | | gggaaaaaggatggaacgtgctggtctggg |
| | | gacgaggttatgcagctgtgaaaaacaggg |
| | | acactgataaggttatttgggtaccctctc |
| | | gaaaagttaaaccggacatcgcccaaaagg |
| | | atgaggtgactaagaaagatgaggcgagcc |
| | | ctctctttgcagaattcgagcccaagaaga |
| | | agaggaaagtc |
| | | (SEQ ID NO: 22) |
| V8 | TVALHLAIPLKWKPNHTPVWIDQWPLPEGK | actgttgcgctacatctggctattccgctc |
| | LVALTQLVEKELQLGHIEPSLSCWNTPVFV | aaatggaagccaaaccacacgcctgtgtgg |
| | IRKASGSYRLLHDLRAVNAKLVPFGAVQQG | attgaccagtggccccttcctgaaggtaaa |
| | APVLSALPRGWPLMVLDLKDCFFSIPLAEQ | cttgtagcgctaacgcaattagtggaaaaa |
| | DREAFAFTLPSVNNQAPARRFQWKVLPQGM | gaattacagttaggacatatagaaccttca |
| | TCSPTICQLIVGQILEPLRLKHPSLRMLHY | cttagttgctggaacacacctgtctttgtg |
| | MDDLLLAASSHDGLEAAGEEVISTLERAGF | atccggaaggcttccgggtcttatcgctta |
| | TISPDKVQREPGVQYLGYKLGSTYVAPVGL | ttgcatgacttgcgcgctgttaacgctaag |
| | VAEPRIATLWDVQKLVGSLQWLRPALGIPP | cttgttccttttggggccgtccaacagggg |
| | RLMGPFYEQLRGSDPNEAREWNLDMKMAWR | gcgccggttctctccgcgctcccgcgtggc |
| | EIVQLSTTAALERWDPALPLEGAVARCEQG | tggcccctgatggtcctagacctcaaggat |
| | AIGVLGQGLSTHPRPCLWLFSTQPTKAFTA | tgcttcttttctattcctcttgcggaacaa |
| | WLEVLTLLITKLRASAVRTFGKEVDILLLP | gatcgcgaagcttttgcatttacgctcccc |
| | ACFREDLPLPEGILLALRGFAGKIRSSDTP | tctgtgaataaccaggcccccgctcgaaga |
| | SIFDIARPLHVSLKVRVTDHPVPGPTVFTD | ttccaatggaaggtcttgccccaagggatg |
| | ASSSTHKGVVVWREGPRWEIKEIADLGASV | acctgttctcccactatctgtcagttgata |
| | QQLEARAVAMALLLWPTTPTNVVTDSAFVA | gtgggtcaaatacttgagcccttgcgactc |
| | KMLLKMGQEGVPSTAAAFILEDALSQRSAM | aagcacccatctctgcgcatgttgcattat |
| | AAVLHVRSHSEVPGFFTEGNDVADSQATFQ | atggatgatcttttgctagccgcctcaagt |
| | AYPLREAKDLHTALHIGPRALSKACNISMQ | catgatgggttggaagcggcaggggaggag |
| | QAREVVQTCPHCNSAPALEAGVNPRGLGPL | gttatcagtacattggaaagagccgggttc |
| | QIWQTDFTLEPRMAPRSWLAVTVDTASSAI | accatttcgcctgataaggtccagagggag |
| | VVTQHGRVTSVAAQHHWATAIAVLGRPKAI | cccggagtacaatatcttgggtacaagtta |
| | KTDNGSCFTSKSTREWLARWGIAHTTGIPG | ggcagtacgtatgtagcacccgtaggcctg |
| | NSQGQAMVERANRLLKDKIRVLAEGDGFMK | gtagcagaacccaggatagccaccttgtgg |
| | RIPTSKQGELLAKAMYALNHFERGENTKTP | gatgttcagaagctggtggggtcacttcag |
| | IQKHWRPTVLTEGPPVKIRIETGEWEKGWN | tggcttcgcccagcgctaggaatcccgcct |
| | VLVWGRGYAAVKNRDTDKVIWVPSRKVKPD | cgactgatgggccccttttatgagcagtta |
| | IAQKDEVTKKDEASPLFAGSGATNFSLLKQ | cgagggtcagatcctaacgaggcgagggaa |
| | AGDVEENPGPTVALHLAIPLKWKPNHTPVW | tggaatctagacatgaaaatggcctggaga |
| | IDQWPLPEGKLVALTQLVEKELQLGHIEPS | gagatcgtgcagctcagcaccactgctgcc |
| | LSCWNTPVFVIRKASGSYRLLHDLRAVNAK | ttggaacgatgggaccctgccctgcctctg |
| | LVPFGAVQQGAPVLSALPRGWPLMVLDLKD | gaaggagcggtcgctagatgtgaacagggg |
| | CFFSIPLAEQDREAFAFTLPSVNNQAPARR | gcaataggggtcctgggacagggactgtcc |
| | FQWKVLPQGMTCSPTICQLIVGQILEPLRL | acacacccaaggccatgtttgtggttattc |
| | KHPSLRMLHYMDDLLLAASSHDGLEAAGEE | tccacccaacccaccaaggcgtttactgct |
| | VISTLERAGFTISPDKVQREPGVQYLGYKL | tggttagaagtgctcacccttttgattact |
| | GSTYVAPVGLVAEPRIATLWDVQKLVGSLQ | aagctacgtgcttcggcagtgcgaaccttt |
| | WLRPALGIPPRLMGPFYBQLRGSDPNEARE | ggcaaggaggttgatatcctcctgttgcct |
| | WNLDMKMAWREIVQLSTTAALERWDPALPL | gcatgctttcgggaggaccttccgctcccg |
| | EGAVARCEQGAIGVLGQGLSTHPRPCLWLF | gaggggatcctgttagcccttagggggttt |
| | STQPTKAFTAWLEVLTLLITKLRASAVRTF | gcaggaaaaatcaggagtagtgacacgcca |
| | GKEVDILLLPACFREDLPLPEGILLALRGF | tctatttttgacattgcgcgtccactgcat |
| | AGKIRSSDTPSIFDIARPLHVSLKVRVTDH | gtttctctgaaagtgagggttaccgaccac |
| | PVPGPTVFTDASSSTHKGVVVWREGPRWEI | cctgtaccgggacccactgtctttaccgac |
| | KEIADLGASVQQLEARAVAMALLLWPTTPT | gcctcctcaagcacccataagggggtggta |
| | NVVTDSAFVAKMLLKMGQEGVPSTAAAFIL | gtctggagggagggcccaaggtgggagata |
| | EDALSQRSAMAAVLHVRSHSEVPGFFTEGN | aaagaaatagctgatttgggggcaagtgta |
| | DVADSQATFQAY | caacaactggaagcacgcgctgtggccatg |
| | (SEQ ID NO: 3) | gcacttctgctgtggccgacaacgcccact |
| | | aatgtagtgactgactctgcgtttgttgcg |
| | | aaaatgttactcaagatggggcaggaggga |
| | | gtcccgtctacagcggcggcttttatttta |
| | | gaggatgcgttaagccaaaggtcagccatg |
| | | gccgccgttctccacgtgcggagtcattct |
| | | gaagtgccagggtttttcacagaaggaaat |
| | | gacgtggcagatagccaagccacctttcaa |
| | | gcgtatcccttgagagaggctaaagatctc |
| | | cataccgctctccatatcggaccccgcgcg |
| | | ctatccaaagcgtgtaatatatctatgcag |
| | | caggctagggaggttgttcagacctgcccg |
| | | cattgtaattcagcccctgcgttggaggcc |
| | | ggggtaaaccctaggggtttgggaccccta |
| | | cagatatggcagacagactttacactagag |
| | | cctagaatggctccccgttcctggctcgct |
| | | gttactgtggataccgcctcatctgcgata |
| | | gtcgtaactcagcatggccgtgtcacatcg |
| | | gttgctgcacaacatcattgggccacggct |
| | | atcgccgttttgggaagaccaaaggccata |
| | | aaaacagataatgggtcctgcttcacgtct |
| | | aaatccacgcgagagtggctcgcgagatgg |
| | | gggatagcacacaccaccgggattccgggt |
| | | aattcccagggtcaagctatggtagagcgg |
| | | gccaaccggctcctgaaagataagatccgt |
| | | gtgcttgcggagggggatggctttatgaaa |
| | | agaatccccaccagcaaacagggggaacta |
| | | ttagccaaggcaatgtatgcccttaatcac |
| | | tttgagcgtggtgaaaacacaaaaacaccg |
| | | atacaaaaacactggagacctaccgttctt |
| | | acagaaggacccccggttaaaatacgaata |
| | | gagacaggggagtgggaaaaaggatggaac |
| | | gtgctggtctggggacgaggttatgcagct |
| | | gtgaaaaacagggacactgataaggttatt |
| | | tgggtaccctctcgaaaagttaaaccggac |
| | | atcgcccaaaaggatgaggtgactaagaaa |
| | | gatgaggcgagccctctctttgcaGGCTCT |
| | | GGAGCCACAAATTTCAGTTTGCTCAAGCAG |
| | | GCTGGAGACGTCGAAGAgAAtCCCGGGCCA |
| | | ACTGTGGCTCTGCATCTGGCCATCCCCCTT |
| | | AAGTGGAAACCCAATCATACACCAGTGTGG |
| | | ATTGACCAATGGCCCCTCCCCGAGGGGAAG |
| | | CTGGTAGCCCTGACACAGCTGGTCGAGAAG |
| | | GAGCTCCAGCTCGGGCATATCGAACCCTCT |
| | | TTGTCCTGTTGGAACACACCTGTCTTTGTC |
| | | ATTAGAAAGGCAAGTGGCTCCTATCGCCTC |
| | | TTGCACGATTTGAGAGCCGTGAACGCCAAA |
| | | CTCGTACCCTTCGGTGCAGTGCAGCAAGGT |
| | | GCCCCCGTATTGAGCGCTCTTCCTCGCGGA |
| | | TGGCCTCTGATGGTACTCGACCTCAAAGAC |
| | | TGTTTTTTCAGTATACCCCTCGCCGAGCAA |
| | | GACAGAGAGGCATTCGCCTTCACCTTGCCA |
| | | TCCGTAAATAATCAGGCCCCAGCCCGGAGG |
| | | TTCCAATGGAAAGTCCTGCCTCAGGGTATG |
| | | ACTTGCAGCCCAACAATTTGCCAGCTTATT |
| | | GTGGGACAAATCCTTGAACCCTTGAGACTT |
| | | AAACACCCAAGTCTCAGGATGCTCCACTAC |
| | | ATGGACGACCTGCTTCTTGCTGCATCCAGC |
| | | CATGATGGACTTGAGGCCGCCGGCGAAGAA |
| | | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAGAT |
| | | GCTTCATCTTCAACCCACAAAGGCGTAGTG |
| | | GTTTGGCGAGAGGGCCCCAGGTGGGAGATA |
| | | AAAGAAATAGCCGACCTCGGGGCAAGCGTT |
| | | CAACAGCTTGAGGCCAGAGCAGTGGCTATG |
| | | GCCTTGCTGCTGTGGCCTACTACTCCCACC |
| | | AATGTTGTGACAGATTCTGCCTTCGTCGCA |
| | | AAGATGCTGCTGAAGATGGGCCAAGAGGGC |
| | | GTTCCTTCAACCGCTGCTGCATTTATTCTC |
| | | GAGGATGCTCTTAGCCAAAGGTCCGCCATG |
| | | GCAGCCGTTCTTCACGTGCGGTCACATTCT |
| | | GAAGTGCCAGGATTCTTCACTGAGGGCAAT |
| | | GATGTCGCAGACTCCCAAGCAACCTTCCAG |
| | | GCATATgaattcgagcccaagaagaagagg |
| | | aaagtc |
| | | (SEQ ID NO: 23) |
| V9 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLFSTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFTD | TTCCAATGGAAAGTCCTGCCTCAGGGTATG |
| | ASSSTHKGVVVWREGPRWEIKEIADLGASV | ACTTGCAGCCCAACAATTTGCCAGCTTATT |
| | QQLEARAVAMALLLWPTTPTNVVTDSAFVA | GTGGGACAAATCCTTGAACCCTTGAGACTT |
| | KMLLKMGQEGVPSTAAAFILEDALSQRSAM | AAACACCCAAGTCTCAGGATGCTCCACTAC |
| | AAVLHVRSHSEVPGFFTEGNDVADSQATFQ | ATGGACGACCTGCTTCTTGCTGCATCCAGC |
| | AY | CATGATGGACTTGAGGCCGCCGGCGAAGAA |
| | (SEQ ID NO: 4) | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAGAT |
| | | GCTTCATCTTCAACCCACAAAGGCGTAGTG |
| | | GTTTGGCGAGAGGGCCCCAGGTGGGAGATA |
| | | AAAGAAATAGCCGACCTCGGGGCAAGCGTT |
| | | CAACAGCTTGAGGCCAGAGCAGTGGCTATG |
| | | GCCTTGCTGCTGTGGCCTACTACTCCCACC |
| | | AATGTTGTGACAGATTCTGCCTTCGTCGCA |
| | | AAGATGTTGCTGAAGATGGGCCAAGAGGGC |
| | | GTTCCTTCAACCGCTGCTGCATTTATTCTC |
| | | GAGGATGCTCTTAGCCAAAGGTCCGCCATG |
| | | GCAGCCGTTCTTCACGTGCGGTCACATTCT |
| | | GAAGTGCCAGGATTCTTCACTGAGGGCAAT |
| | | GATGTCGCAGACTCCCAAGCAACCTTCCAG |
| | | GCATATgaattcgagcccaagaagaagagg |
| | | aaagtc |
| | | (SEQ ID NO: 24) |
| V10 | TVALHLAIPLKWKPNHTPVWIDQWPLPEGK | actgttgcgctacatctggctattccgctc |
| | LVALTQLVEKELQLGHIEPSLSCWNTPVFV | aaatggaagccaaaccacacgcctgtgtgg |
| | IRKASGSYRLLHDLRAVNAKLVPFGAVQQG | attgaccagtggccccttcctgaaggtaaa |
| | APVLSALPRGWPLMVLDLKDCFFSIPLAEQ | cttgtagcgctaacgcaattagtggaaaaa |
| | DREAFAFTLPSVNNQAPARRFQWKVLPQGM | gaattacagttaggacatatagaaccttca |
| | TCSPTICQLIVGQILEPLRLKHPSLRMLHY | cttagttgctggaacacacctgtctttgtg |
| | MDDLLLAASSHDGLEAAGEEVISTLERAGF | atccggaaggcttccgggtcttatcgctta |
| | TISPDKVQREPGVQYLGYKLGSTYVAPVGL | ttgcatgacttgcgcgctgttaacgctaag |
| | VAEPRIATLWDVQKLVGSLQWLRPALGIPP | cttgttccttttggggccgtccaacagggg |
| | RLMGPFYEQLRGSDPNEAREWNLDMKMAWR | gcgccggttctctccgcgctcccgcgtggc |
| | EIVQLSTTAALERWDPALPLEGAVARCEQG | tggcccctgatggtcctagacctcaaggat |
| | AIGVLGQGLSTHPRPCLWLFSTQPTKAFTA | tgcttcttttctattcctcttgcggaacaa |
| | WLEVLTLLITKLRASAVRTFGKEVDILLLP | gatcgcgaagcttttgcatttacgctcccc |
| | ACFREDLPLPEGILLALRGFAGKIRSSDTP | tctgtgaataaccaggcccccgctcgaaga |
| | SIFDIARPLHVSLKVRVTDHPVPGPTVFTD | ttccaatggaaggtcttgccccaagggatg |
| | ASSSTHKGVVVWREGPRWEIKEIADLGASV | acctgttctcccactatctgtcagttgata |
| | QQLEARAVAMALLLWPTTPTNVVTDSAFVA | gtgggtcaaatacttgagcccttgcgactc |
| | KMLLKMGQEGVPSTAAAFILEDALSQRSAM | aagcacccatctctgcgcatgttgcattat |
| | AAVLHVRSHSEVPGFFTEGNDVADSQATFQ | atggatgatcttttgctagccgcctcaagt |
| | AYPLREAKDLHTALHIGPRALSKACNISMQ | catgatgggttggaagcggcaggggaggag |
| | QAREVVQTCPHCNSAPALEAGVNPRGLGPL | gttatcagtacattggaaagagccgggttc |
| | QIWQTDFTLEPRMAPRSWLAVTVDTASSAI | accatttcgcctgataaggtccagagggag |
| | VVTQHGRVTSVAAQHHWATAIAVLGRPKAI | cccggagtacaatatcttgggtacaagtta |
| | KTDNGSCFTSKSTREWLARWGIAHTTGIPG | ggcagtacgtatgtagcacccgtaggcctg |
| | NSQGQAMVERANRLLKDKIRVLAEGDGFMK | gtagcagaacccaggatagccaccttgtgg |
| | RIPTSKQGELLAKAMYALNHFERGENTKTP | gatgttcagaagctggtggggtcacttcag |
| | IQKHWRPTVLTEGPPVKIRIETGEWEKGWN | tggcttcgcccagcgctaggaatcccgcct |
| | VLVWGRGYAAVKNRDTDKVIW | cgactgatgggccccttttatgagcagtta |
| | (SEQ ID NO: 5) | cgagggtcagatcctaacgaggcgagggaa |
| | | tggaatctagacatgaaaatggcctggaga |
| | | gagatcgtgcagctcagcaccactgctgcc |
| | | ttggaacgatgggaccctgccctgcctctg |
| | | gaaggagcggtcgctagatgtgaacagggg |
| | | gcaataggggtcctgggacagggactgtcc |
| | | acacacccaaggccatgtttgtggttattc |
| | | tccacccaacccaccaaggcgtttactgct |
| | | tggttagaagtgctcacccttttgattact |
| | | aagctacgtgcttcggcagtgcgaaccttt |
| | | ggcaaggaggttgatatcctcctgttgcct |
| | | gcatgctttcgggaggaccttccgctcccg |
| | | gaggggatcctgttagcccttagggggttt |
| | | gcaggaaaaatcaggagtagtgacacgcca |
| | | tctatttttgacattgcgcgtccactgcat |
| | | gtttctctgaaagtgagggttaccgaccac |
| | | cctgtaccgggacccactgtctttaccgac |
| | | gcctcctcaagcacccataagggggtggta |
| | | gtctggagggagggcccaaggtgggagata |
| | | aaagaaatagctgatttgggggcaagtgta |
| | | caacaactggaagcacgcgctgtggccatg |
| | | gcacttctgctgtggccgacaacgcccact |
| | | aatgtagtgactgactctgcgtttgttgcg |
| | | aaaatgttactcaagatggggcaggaggga |
| | | gtcccgtctacagcggcggcttttatttta |
| | | gaggatgcgttaagccaaaggtcagccatg |
| | | gccgccgttctccacgtgcggagtcattct |
| | | gaagtgccagggtttttcacagaaggaaat |
| | | gacgtggcagatagccaagccacctttcaa |
| | | gcgtatcccttgagagaggctaaagatctc |
| | | cataccgctctccatatcggaccccgcgcg |
| | | ctatccaaagcgtgtaatatatctatgcag |
| | | caggctagggaggttgttcagacctgcccg |
| | | cattgtaattcagcccctgcgttggaggcc |
| | | ggggtaaaccctaggggtttgggaccccta |
| | | cagatatggcagacagactttacactagag |
| | | cctagaatggctccccgttcctggctcgct |
| | | gttactgtggataccgcctcatctgcgata |
| | | gtcgtaactcagcatggccgtgtcacatcg |
| | | gttgctgcacaacatcattgggccacggct |
| | | atcgccgttttgggaagaccaaaggccata |
| | | aaaacagataatgggtcctgcttcacgtct |
| | | aaatccacgcgagagtggctcgcgagatgg |
| | | gggatagcacacaccaccgggattccgggt |
| | | aattcccagggtcaagctatggtagagcgg |
| | | gccaaccggctcctgaaagataagatccgt |
| | | gtgcttgcggagggggatggctttatgaaa |
| | | agaatccccaccagcaaacagggggaacta |
| | | ttagccaaggcaatgtatgcccttaatcac |
| | | tttgagcgtggtgaaaacacaaaaacaccg |
| | | atacaaaaacactggagacctaccgttctt |
| | | acagaaggacccccggttaaaatacgaata |
| | | gagacaggggagtgggaaaaaggatggaac |
| | | gtgctggtctggggacgaggttatgcagct |
| | | gtgaaaaacagggacactgataaggttatt |
| | | tgggtaccctctcgaaaagttaaaccggac |
| | | atcgcccaaaaggatgaggtgactaagaaa |
| | | gatgaggcgagccctctctttgcagaattc |
| | | gagcccaagaagaagaggaaagtc |
| | | (SEQ ID NO: 25) |
| V11 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLESTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFTD | TTCCAATGGAAAGTCCTGCCTCAGGGTATG |
| | ASSSTHKGVVVWREGPRWEIKEIADLGASV | ACTTGCAGCCCAACAATTTGCCAGCTTATT |
| | QQLEARAVAMALLLWPTTPTNVVTDSAFVA | GTGGGACAAATCCTTGAACCCTTGAGACTT |
| | KMLLKMGQEGVPSTAAAFILEDALSQRSAM | AAACACCCAAGTCTCAGGATGCTCCACTAC |
| | AAVLHVRSHSEVPGFFTEGNDVADSQATFQ | ATGGACGACCTGCTTCTTGCTGCATCCAGC |
| | AY | CATGATGGACTTGAGGCCGCCGGCGAAGAA |
| | (SEQ ID NO: 6) | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAGAT |
| | | GCTTCATCTTCAACCCACAAAGGCGTAGTG |
| | | GTTTGGCGAGAGGGCCCCAGGTGGGAGATA |
| | | AAAGAAATAGCCGACCTCGGGGCAAGCGTT |
| | | CAACAGCTTGAGGCCAGAGCAGTGGCTATG |
| | | GCCTTGCTGCTGTGGCCTACTACTCCCACC |
| | | AATGTTGTGACAGATTCTGCCTTCGTCGCA |
| | | AAGATGTTGCTGAAGATGGGCCAAGAGGGC |
| | | GTTCCTTCAACCGCTGCTGCATTTATTCTC |
| | | GAGGATGCTCTTAGCCAAAGGTCCGCCATG |
| | | GCAGCCGTTCTTCACGTGCGGTCACATTCT |
| | | GAAGTGCCAGGATTCTTCACTGAGGGCAAT |
| | | GATGTCGCAGACTCCCAAGCAACCTTCCAG |
| | | GCATAT |
| | | (SEQ ID NO: 26) |
| V12 | TVALHLAIPLKWKPNHTPVWIDQWPLPEGK | actgttgcgctacatctggctattccgctc |
| | LVALTQLVEKELQLGHIEPSLSCWNTPVFV | aaatggaagccaaaccacacgcctgtgtgg |
| | IRKASGSYRLLHDLRAVNAKLVPFGAVQQG | attgaccagtggccccttcctgaaggtaaa |
| | APVLSALPRGWPLMVLDLKDCFFSIPLAEQ | cttgtagcgctaacgcaattagtggaaaaa |
| | DREAFAFTLPSVNNQAPARRFQWKVLPQGM | gaattacagttaggacatatagaaccttca |
| | TCSPTICQLIVGQILEPLRLKHPSLRMLHY | cttagttgctggaacacacctgtctttgtg |
| | MDDLLLAASSHDGLEAAGEEVISTLERAGF | atccggaaggcttccgggtcttatcgctta |
| | TISPDKVQREPGVQYLGYKLGSTYVAPVGL | ttgcatgacttgcgcgctgttaacgctaag |
| | VAEPRIATLWDVQKLVGSLQWLRPALGIPP | cttgttccttttggggccgtccaacagggg |
| | RLMGPFYEQLRGSDPNEAREWNLDMKMAWR | gcgccggttctctccgcgctcccgcgtggc |
| | EIVQLSTTAALERWDPALPLEGAVARCEQG | tggcccctgatggtcctagacctcaaggat |
| | AIGVLGQGLSTHPRPCLWLESTQPTKAFTA | tgcttcttttctattcctcttgcggaacaa |
| | WLEVLTLLITKLRASAVRTFGKEVDILLLP | gatcgcgaagcttttgcatttacgctcccc |
| | ACFREDLPLPEGILLALRGFAGKIRSSDTP | tctgtgaataaccaggcccccgctcgaaga |
| | SIFDIARPLHVSLKVRVTDHPVPGPTVFTD | ttccaatggaaggtcttgccccaagggatg |
| | ASSSTHKGVVVWREGPRWEIKEIADLGASV | acctgttctcccactatctgtcagttgata |
| | QQLEARAVAMALLLWPTTPTNVVTDSAFVA | gtgggtcaaatacttgagcccttgcgactc |
| | KMLLKMGQEGVPSTAAAFILEDALSQRSAM | aagcacccatctctgcgcatgttgcattat |
| | AAVLHVRSHSEVPGFFTEGNDVADSQATFQ | atggatgatcttttgctagccgcctcaagt |
| | AYPLREAKDLHTALHIGPRALSKACNISMQ | catgatgggttggaagcggcaggggaggag |
| | QAREVVQTCPHCNSAPALEAGVNPRGLGPL | gttatcagtacattggaaagagccgggttc |
| | QIWQTDFTLEPRMAPRSWLAVTVDTASSAI | accatttcgcctgataaggtccagagggag |
| | VVTQHGRVTSVAAQHHWATAIAVLGRPKAI | cccggagtacaatatcttgggtacaagtta |
| | KTDNGSCFTSKSTREWLARWGIAHTTGIPG | ggcagtacgtatgtagcacccgtaggcctg |
| | NSQGQAMVERANRLLKDKIRVLAEGDGFMK | gtagcagaacccaggatagccaccttgtgg |
| | RIPTSKQGELLAKAMYALNHFERGENTKTP | gatgttcagaagctggtggggtcacttcag |
| | IQKHWRPTVLTEGPPVKIRIETGEWEKGWN | tggcttcgcccagcgctaggaatcccgcct |
| | VLVWGRGYAAVKNRDTDKVIWVPSRKVKPD | cgactgatgggccccttttatgagcagtta |
| | IAQKDEVTKKDEASPLFA | cgagggtcagatcctaacgaggcgagggaa |
| | (SEQ ID NO: 7) | tggaatctagacatgaaaatggcctggaga |
| | | gagatcgtgcagctcagcaccactgctgcc |
| | | ttggaacgatgggaccctgccctgcctctg |
| | | gaaggagcggtcgctagatgtgaacagggg |
| | | gcaataggggtcctgggacagggactgtcc |
| | | acacacccaaggccatgtttgtggttattc |
| | | tccacccaacccaccaaggcgtttactgct |
| | | tggttagaagtgctcacccttttgattact |
| | | aagctacgtgcttcggcagtgcgaaccttt |
| | | ggcaaggaggttgatatcctcctgttgcct |
| | | gcatgctttcgggaggaccttccgctcccg |
| | | gaggggatcctgttagcccttagggggttt |
| | | gcaggaaaaatcaggagtagtgacacgcca |
| | | tctatttttgacattgcgcgtccactgcat |
| | | gtttctctgaaagtgagggttaccgaccac |
| | | cctgtaccgggacccactgtctttaccgac |
| | | gcctcctcaagcacccataagggggtggta |
| | | gtctggagggagggcccaaggtgggagata |
| | | aaagaaatagctgatttgggggcaagtgta |
| | | caacaactggaagcacgcgctgtggccatg |
| | | gcacttctgctgtggccgacaacgcccact |
| | | aatgtagtgactgactctgcgtttgttgcg |
| | | aaaatgttactcaagatggggcaggaggga |
| | | gtcccgtctacagcggcggcttttatttta |
| | | gaggatgcgttaagccaaaggtcagccatg |
| | | gccgccgttctccacgtgcggagtcattct |
| | | gaagtgccagggtttttcacagaaggaaat |
| | | gacgtggcagatagccaagccacctttcaa |
| | | gcgtatcccttgagagaggctaaagatctc |
| | | cataccgctctccatatcggaccccgcgcg |
| | | ctatccaaagcgtgtaatatatctatgcag |
| | | caggctagggaggttgttcagacctgcccg |
| | | cattgtaattcagcccctgcgttggaggcc |
| | | ggggtaaaccctaggggtttgggaccccta |
| | | cagatatggcagacagactttacactagag |
| | | cctagaatggctccccgttcctggctcgct |
| | | gttactgtggataccgcctcatctgcgata |
| | | gtcgtaactcagcatggccgtgtcacatcg |
| | | gttgctgcacaacatcattgggccacggct |
| | | atcgccgttttgggaagaccaaaggccata |
| | | aaaacagataatgggtcctgcttcacgtct |
| | | aaatccacgcgagagtggctcgcgagatgg |
| | | gggatagcacacaccaccgggattccgggt |
| | | aattcccagggtcaagctatggtagagcgg |
| | | gccaaccggctcctgaaagataagatccgt |
| | | gtgcttgcggagggggatggctttatgaaa |
| | | agaatccccaccagcaaacagggggaacta |
| | | ttagccaaggcaatgtatgcccttaatcac |
| | | tttgagcgtggtgaaaacacaaaaacaccg |
| | | atacaaaaacactggagacctaccgttctt |
| | | acagaaggacccccggttaaaatacgaata |
| | | gagacaggggagtgggaaaaaggatggaac |
| | | gtgctggtctggggacgaggttatgcagct |
| | | gtgaaaaacagggacactgataaggttatt |
| | | tgggtaccctctcgaaaagttaaaccggac |
| | | atcgcccaaaaggatgaggtgactaagaaa |
| | | gatgaggcgagccctctctttgca |
| | | (SEQ ID NO: 27) |
| V13 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLESTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFTA | TTCCAATGGAAAGTCCTGCCTCAGGGTATG |
| | ASSSTHKGVVVWREGPRWEIKEIADLGASV | ACTTGCAGCCCAACAATTTGCCAGCTTATT |
| | QQLEARAVAMALLLWPTTPTNVVTDSAFVA | GTGGGACAAATCCTTGAACCCTTGAGACTT |
| | KMLLKMGQEGVPSTAAAFILEDALSQRSAM | AAACACCCAAGTCTCAGGATGCTCCACTAC |
| | AAVLHVRSHSEVPGFFTEGNDVADSQATFQ | ATGGACGACCTGCTTCTTGCTGCATCCAGC |
| | AY | CATGATGGACTTGAGGCCGCCGGCGAAGAA |
| | (SEQ ID NO: 8) | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAGcT |
| | | GCTTCATCTTCAACCCACAAAGGCGTAGTG |
| | | GTTTGGCGAGAGGGCCCCAGGTGGGAGATA |
| | | AAAGAAATAGCCGACCTCGGGGCAAGCGTT |
| | | CAACAGCTTGAGGCCAGAGCAGTGGCTATG |
| | | GCCTTGCTGCTGTGGCCTACTACTCCCACC |
| | | AATGTTGTGACAGATTCTGCCTTCGTCGCA |
| | | AAGATGTTGCTGAAGATGGGCCAAGAGGGC |
| | | GTTCCTTCAACCGCTGCTGCATTTATTCTC |
| | | GAGGATGCTCTTAGCCAAAGGTCCGCCATG |
| | | GCAGCCGTTCTTCACGTGCGGTCACATTCT |
| | | GAAGTGCCAGGATTCTTCACTGAGGGCAAT |
| | | GATGTCGCAGACTCCCAAGCAACCTTCCAG |
| | | GCATATgaattcgagcccaagaagaagagg |
| | | aaagtc |
| | | (SEQ ID NO: 28) |
| V14 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLESTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFTA | TTCCAATGGAAAGTCCTGCCTCAGGGTATG |
| | ASSSTHKGVVVWREGPRWEIKEIADLGASV | ACTTGCAGCCCAACAATTTGCCAGCTTATT |
| | QQLAARAVAMALLLWPTTPTNVVTASAFVA | GTGGGACAAATCCTTGAACCCTTGAGACTT |
| | KMLLKMGQEGVPSTAAAFILEDALSQRSAM | AAACACCCAAGTCTCAGGATGCTCCACTAC |
| | AAVLHVRSHSEVPGFFTEGNDVADSQATFQ | ATGGACGACCTGCTTCTTGCTGCATCCAGC |
| | AY | CATGATGGACTTGAGGCCGCCGGCGAAGAA |
| | (SEQ ID NO: 9) | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAGcT |
| | | GCTTCATCTTCAACCCACAAAGGCGTAGTG |
| | | GTTTGGCGAGAGGGCCCCAGGTGGGAGATA |
| | | AAAGAAATAGCCGACCTCGGGGCAAGCGTT |
| | | CAACAGCTTGcGGCCAGAGCAGTGGCTATG |
| | | GCCTTGCTGCTGTGGCCTACTACTCCCACC |
| | | AATGTTGTGACAGETTCTGCCTTCGTCGCA |
| | | AAGATGTTGCTGAAGATGGGCCAAGAGGGC |
| | | GTTCCTTCAACCGCTGCTGCATTTATTCTC |
| | | GAGGATGCTCTTAGCCAAAGGTCCGCCATG |
| | | GCAGCCGTTCTTCACGTGCGGTCACATTCT |
| | | GAAGTGCCAGGATTCTTCACTGAGGGCAAT |
| | | GATGTCGCAGACTCCCAAGCAACCTTCCAG |
| | | GCATATgaattcgagcccaagaagaagagg |
| | | aaagtc |
| | | (SEQ ID NO: 29) |
| V15 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERW | TGGCCTCTGATGGTACTCGACCTCAAAGAC |
| | (SEQ ID NO: 10) | TGTTTTTTCAGTATACCCCTCGCCGAGCAA |
| | | GACAGAGAGGCATTCGCCTTCACCTTGCCA |
| | | TCCGTAAATAATCAGGCCCCAGCCCGGAGG |
| | | TTCCAATGGAAAGTCCTGCCTCAGGGTATG |
| | | ACTTGCAGCCCAACAATTTGCCAGCTTATT |
| | | GTGGGACAAATCCTTGAACCCTTGAGACTT |
| | | AAACACCCAAGTCTCAGGATGCTCCACTAC |
| | | ATGGACGACCTGCTTCTTGCTGCATCCAGC |
| | | CATGATGGACTTGAGGCCGCCGGCGAAGAA |
| | | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGgaattcgagcccaagaag |
| | | aagaggaaagtc |
| | | (SEQ ID NO: 30) |
| V16 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLFSTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SI | TTCCAATGGAAAGTCCTGCCTCAGGGTATG |
| | (SEQ ID NO: 11) | ACTTGCAGCCCAACAATTTGCCAGCTTATT |
| | | GTGGGACAAATCCTTGAACCCTTGAGACTT |
| | | AAACACCCAAGTCTCAGGATGCTCCACTAC |
| | | ATGGACGACCTGCTTCTTGCTGCATCCAGC |
| | | CATGATGGACTTGAGGCCGCCGGCGAAGAA |
| | | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCgaattcgagcccaagaagaagagg |
| | | aaagtc |
| | | (SEQ ID NO: 31) |
| V17 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLFSTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFT | TTCCAATGGAAAGTCCTGCCTCAGGGTATG |
| | (SEQ ID NO: 12) | ACTTGCAGCCCAACAATTTGCCAGCTTATT |
| | | GTGGGACAAATCCTTGAACCCTTGAGACTT |
| | | AAACACCCAAGTCTCAGGATGCTCCACTAC |
| | | ATGGACGACCTGCTTCTTGCTGCATCCAGC |
| | | CATGATGGACTTGAGGCCGCCGGCGAAGAA |
| | | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAgaa |
| | | ttcgagcccaagaagaagaggaaagtc |
| | | (SEQ ID NO: 32) |
| V18 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALELAENREILKEPVHGVYYD | TGGCCTCTGATGGTACTCGACCTCAAAGAC |
| | PSKDLIAEIQKQGQGQWTYQIYQEPFKNLK | TGTTTTTTCAGTATACCCCTCGCCGAGCAA |
| | TGKYARMRGAHTNDVKQLTEAVQKITTESI | GACAGAGAGGCATTCGCCTTCACCTTGCCA |
| | VIWGKTPKFKLPIQKETWETWWTEYWQATW | TCCGTAAATAATCAGGCCCCAGCCCGGAGG |
| | IPEWEFVNTPPLVKLWYQLEKEPIVGAET | TTCCAATGGAAAGTCCTGCCTCAGGGTATG |
| | (SEQ ID NO: 13) | ACTTGCAGCCCAACAATTTGCCAGCTTATT |
| | | GTGGGACAAATCCTTGAACCCTTGAGACTT |
| | | AAACACCCAAGTCTCAGGATGCTCCACTAC |
| | | ATGGACGACCTGCTTCTTGCTGCATCCAGC |
| | | CATGATGGACTTGAGGCCGCCGGCGAAGAA |
| | | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACTGGCAGAAAACAGAGAGATTCTA |
| | | AAAGAACCAGTACATGGAGTGTATTATGAC |
| | | CCATCAAAAGACTTAATAGCAGAAATACAG |
| | | AAGCAGGGGCAAGGCCAATGGACATATCAA |
| | | ATTTATCAAGAGCCATTTAAAAATCTGAAA |
| | | ACAGGAAAATATGCAAGAATGAGGGGTGCC |
| | | CACACTAATGATGTAAAACAATTAACAGAG |
| | | GCAGTGCAAAAAATAACCACAGAAAGCATA |
| | | GTAATATGGGGAAAGACTCCTAAATTTAAA |
| | | CTGCCCATACAAAAGGAAACATGGGAAACA |
| | | TGGTGGACAGAGTATTGGCAAGCCACCTGG |
| | | ATTCCTGAGTGGGAGTTTGTTAATACCCCT |
| | | CCCTTAGTGAAATTATGGTACCAGTTAGAG |
| | | AAAGAACCCATAGTAGGAGCAGAAACCTTC |
| | | gaattcgagcccaagaagaagaggaaagtc |
| | | (SEQ ID NO: 33) |
| V19 | TVALHLAIPLKWKPDHTPVWIDQWPLPEGK | ACTGTTGCGCTACATCTGGCTATTCCGCTC |
| | LVALTQLVEKELQLGHIEPSLSCWNTPVFV | AAATGGAAGCCAGACCACACGCCTGTGTGG |
| | IRKASGSYRLLHDLRAVNAKLVPFGAVQQG | ATTGACCAGTGGCCCCTCCCTGAAGGTAAA |
| | APVLSALPRGWPLMVLDLKDCFFSIPLAEQ | CTTGTAGCGCTAACGCAATTAGTGGAAAAA |
| | DREAFAFTLPSVNNQAPARRFQWKVLPQGM | GAATTACAGTTAGGACATATAGAACCTTCA |
| | TCSPTICQLVVGQVLEPLRLKHPSLCMLHY | CTTAGTTGTTGGAACACACCTGTCTTCGTG |
| | MDDLLLAASSHDGLEAAGEEVISTLERAGF | ATCCGGAAGGCTTCCGGGTCTTACCGCTTA |
| | TISPDKVQREPGVQYLGYKLGSTYVAPVGL | CTGCATGATTTGCGCGCTGTTAACGCCAAG |
| | VAEPRIATLWDVQKLVGSLQWLRPALGIPP | CTTGTTCCTTTTGGGGCCGTCCAACAGGGG |
| | RLMGPFYEQLRGSDPNEAREWNLDMKMAWR | GCGCCAGTTCTCTCCGCGCTCCCGCGTGGC |
| | EIVRLSTTAALERWDPALPLEGAVARCEQG | TGGCCCCTGATGGTCTTAGACCTCAAGGAT |
| | AIGVLGQGLSTHPRPCLWLFSTQPTKAFTA | TGCTTCTTTTCTATCCCTCTTGCGGAACAA |
| | WLEVLTLLITKLRASAVRTFGKEVDILLLP | GATCGCGAAGCTTTTGCATTTACGCTCCCC |
| | ACFREDLPLPEGILLALKGFAGKIRSSDTP | TCTGTGAATAACCAGGCCCCCGCTCGAAGA |
| | SIFDIARPLHVSLKVRVTDHPVPGPTVFTD | TTCCAATGGAAGGTCTTGCCCCAAGGGATG |
| | ASSSTHKGVVVWREGPRWEIKEIADLGASV | ACCTGTTCTCCCACTATCTGTCAGTTGGTA |
| | QQLEARAVAMALLLWPTTPTNVVTNSAFVA | GTGGGTCAGGTACTTGAGCCCTTGCGACTC |
| | KMLLKMGQEGVPSTAAAFILEDALSQRSAM | AAGCACCCATCTCTGTGCATGTTGCATTAT |
| | AAVLHVRSHSEVPGFFTEGNDVADSQATFQ | ATGGATGATCTTTTGCTAGCCGCCTCAAGT |
| | AY | CACGATGGGTTGGAAGCGGCAGGGGAGGAG |
| | (SEQ ID NO: 14) | GTTATCAGTACATTGGAAAGAGCCGGGTTC |
| | | ACTATTTCGCCTGATAAGGTCCAGAGGGAG |
| | | CCCGGAGTACAATATCTTGGGTACAAGTTA |
| | | GGCAGTACGTATGTAGCACCCGTAGGCCTG |
| | | GTAGCAGAACCCAGGATAGCCACCTTGTGG |
| | | GATGTTCAAAAGCTGGTGGGGTCACTTCAG |
| | | TGGCTTCGCCCAGCGTTAGGAATCCCGCCA |
| | | CGACTGATGGGCCCCTTCTATGAGCAGTTA |
| | | CGAGGGTCAGATCCTAACGAGGCGAGGGAA |
| | | TGGAATCTAGACATGAAAATGGCCTGGAGA |
| | | GAGATCGTACGGCTTAGCACCACTGCTGCC |
| | | TTGGAACGATGGGACCCTGCCCTGCCTCTG |
| | | GAAGGAGCGGTCGCTAGATGTGAACAGGGG |
| | | GCAATAGGGGTTTTGGGACAGGGACTGTCC |
| | | ACACACCCAAGGCCATGCTTGTGGTTATTC |
| | | TCCACCCAACCCACCAAGGCGTTTACTGCT |
| | | TGGTTAGAAGTGCTCACCCTTTTGATTACT |
| | | AAGCTACGTGCTTCGGCAGTGCGAACCTTT |
| | | GGCAAGGAGGTCGATATCCTCCTGTTGCCT |
| | | GCATGCTTTCGGGAGGACCTTCCGCTCCCA |
| | | GAGGGGATCCTGTTAGCCCTTAAGGGGTTT |
| | | GCAGGAAAAATCAGGAGTAGTGACACGCCA |
| | | TCTATTTTTGACATTGCGCGTCCACTGCAT |
| | | GTTTCTCTGAAAGTGAGGGTTACCGACCAC |
| | | CCTGTGCCGGGACCCACTGTCTTTACTGAC |
| | | GCCTCCTCAAGCACCCATAAGGGGGTGGTA |
| | | GTCTGGAGGGAGGGCCCAAGGTGGGAGATA |
| | | AAAGAAATAGCTGATTTGGGGGCAAGTGTA |
| | | CAACAACTGGAAGCACGCGCTGTGGCCATG |
| | | GCACTTCTGCTGTGGCCGACAACGCCCACT |
| | | AATGTAGTGACTAACTCCGCGTTTGTTGCG |
| | | AAAATGTTACTCAAGATGGGACAGGAGGGA |
| | | GTCCCGTCTACAGCGGCGGCTTTTATTTTA |
| | | GAGGATGCGTTAAGCCAAAGGTCAGCCATG |
| | | GCCGCCGTTCTCCACGTGCGGAGTCATTCT |
| | | GAAGTGCCAGGGTTTTTCACAGAAGGAAAT |
| | | GACGTGGCAGATAGCCAAGCCACCTTCCAA |
| | | GCGTATgaattcgagcccaagaagaagagg |
| | | aaagtc |
| | | (SEQ ID NO: 34) |
| V20 | MDTSNLMEQILSSDNLNRAYLQVVRNKGAE | atggataccagcaatctgatggaacaaatt |
| | GVDGMKYTELKEHLAKNGETIKGQLRTRKY | ctgagcagcgataatctgaatcgtgcatat |
| | KPQPARRVEIPKPDGGVRNLGVPTVTDRFI | ctgcaggttgtgcgtaataaaggtgccgaa |
| | QQAIAQVLTPIYEEQFHDHSYGFRPNRCAQ | ggtgttgatggtatgaaatataccgaactg |
| | QAILTALNIMNDGNDWIVDIDLEKFFDTVN | aaagaacacctggccaaaaatggtgaaacc |
| | HDKLMTLIGRTIKDGDVISIVRKYLVSGIM | attaaaggtcagctgcgtacccgtaaatac |
| | IDDEYEDSIVGTPQGGNLSPLLANIMLNEL | aaaccgcagcctgcacgtcgtgttgaaatt |
| | DKEMEKRGLNFVRYADDCIIMVGSEMSANR | ccgaaaccggatggtggtgttcgtaatctg |
| | VMRNISRFIEEKLGLKVNMTKSKVDRPSGL | ggtgttccgaccgttaccgatcgttttatt |
| | KYLGFGFYFDPRAHQFKAKPHAKSVAKFKK | cagcaggcaattgcacaggttctgaccccg |
| | RMKELTCRSWGVSNSYKVEKLNQLIRGWIN | atttatgaagaacagtttcatgatcacagc |
| | YFKIGSMKTLCKELDSRIRYRLRMCIWKQW | tatggctttcgtccgaatcgttgtgcacag |
| | KTPQNQEKNLVKLGIDRNTARRVAYTGKRI | caggccattctgaccgcactgaatattatg |
| | AYVCNKGAVNVAISNKRLASFGLISMLDYY | aatgatggcaatgattggatcgtggatatc |
| | IEKCVTC | gacctggaaaaattctttgataccgtgaac |
| | (SEQ ID NO: 15) | cacgataaactgatgaccctgattggtcgt |
| | | acaatcaaagatggtgatgtgattagcatc |
| | | gtgcgtaaatatctggttagcggcattatg |
| | | atcgatgatgaatatgaggatagcattgtt |
| | | ggtacaccgcagggggtaatctgagtccgc |
| | | tgctggccaatattatgctgaatgaactgg |
| | | acaaagagatggaaaaacgcggtctgaatt |
| | | ttgttcgttatgccgatgattgcattatta |
| | | tggttggtagcgaaatgagcgcaaatcgtg |
| | | ttatgcgtaatattagccgcttcatcgaag |
| | | aaaaactgggcctgaaagttaacatgacca |
| | | aaagcaaagttgatcgtccgagtggtctga |
| | | aatacctgggttttggtttctattttgatc |
| | | cgcgtgcacaccagtttaaagcaaaaccgc |
| | | atgcaaaaagcgtggccaaattcaaaaaac |
| | | gcatgaaagaactgacctgtcgtagctggg |
| | | gtgttagcaatagctataaagtggaaaaac |
| | | tgaaccagctgattcgcggttggatcaact |
| | | atttcaaaattggcagcatgaaaaccctgt |
| | | gcaaagaactggatagccgtattcgttatc |
| | | gtctgcgtatgtgtatttggaaacagtgga |
| | | aaacaccgcagaatcaagaaaaaaatctgg |
| | | tgaaactgggcattgatcgtaataccgcac |
| | | gtcgcgttgcatataccggtaaacgtattg |
| | | catatgtgtgcaataaaggcgcagttaatg |
| | | ttgccattagcaataaacgtctggcaagct |
| | | ttggtctgattagtatgctggattactaca |
| | | tcgaaaaatgcgtgacctgcccctctggcg |
| | | gctcaaaaagaaccgccgacggcagcgaat |
| | | tcgagcccaagaagaagaggaaagtc |
| | | (SEQ ID NO: 35) |
| V21 | PISPIETVPVKLKPGMDGPKVKQWPLTEEK | CCCATTAGCCCTATTGAGACTGTACCAGTA |
| | IKALVEICTEMEKEGKISKIGPENPYNTPV | AAATTAAAGCCAGGAATGGATGGCCCAAAA |
| | FAIKKKDSTKWRKLVDFRELNKRTQDFWEV | GTTAAACAATGGCCATTGACAGAAGAAAAA |
| | QLGIPHPAGLKKKKSVTVLDVGDAYFSVPL | ATAAAAGCATTAGTAGAAATTTGTACAGAG |
| | DEDFRKYTAFTIPSINNETPGIRYQYNVLP | ATGGAAAAGGAAGGGAAAATTTCAAAAATT |
| | QGWKGSPAIFQSSMTKILEPFRKQNPDIVI | GGGCCTGAAAATCCATACAATACTCCAGTA |
| | YQYMDDLYVGSDLEIGQHRTKIEELRQHLL | TTTGCCATAAAGAAAAAAGACAGTACTAAA |
| | RWGLTTPDKKHQKEPPFLWMGYELHPDKWT | TGGAGAAAATTAGTAGATTTCAGAGAACTT |
| | VQPIVLPEKDSWTVNDIQKLVGKLNWASQI | AATAAGAGAACTCAAGACTTCTGGGAAGTT |
| | YPGIKVRQLCKLLRGTKALTEVIPLTEEAE | CAATTAGGAATACCACATCCCGCAGGGTTA |
| | LELAENREILKEPVHGVYYDPSKDLIAEIQ | AAAAAGAAAAAATCAGTAACAGTACTGGAT |
| | KQGQGQWTYQIYQEPFKNLKTGKYARMRGA | GTGGGTGATGCATATTTTTCAGTTCCCTTA |
| | HTNDVKQLTEAVQKITTESIVIWGKTPKFK | GATGAAGACTTCAGGAAGTATACTGCATTT |
| | LPIQKETWETWWTEYWQATWIPEWEFVNTP | ACCATACCTAGTATAAACAATGAGACACCA |
| | PLVKLWYQLEKEPIVGAETFYVDGAANRET | GGGATTAGATATCAGTACAATGTGCTTCCA |
| | KLGKAGYVTNRGRQKVVTLTDTTNQKTELQ | CAGGGATGGAAAGGATCACCAGCAATATTC |
| | AIYLALQDSGLEVNIVTDSQYALGIIQAQP | CAAAGTAGCATGACAAAAATCTTAGAGCCT |
| | DQSESELVNQIIEQLIKKEKVYLAWVPAHK | TTTAGAAAACAAAATCCAGACATAGTTATC |
| | GIGGNEQVDKLVSAGIRKVL | TATCAATACATGGATGATTTGTATGTAGGA |
| | (SEQ ID NO: 16) | TCTGACTTAGAAATAGGGCAGCATAGAACA |
| | | AAAATAGAGGAGCTGAGACAACATCTGTTG |
| | | AGGTGGGGACTTACCACACCAGACAAAAAA |
| | | CATCAGAAAGAACCTCCATTCCTTTGGATG |
| | | GGTTATGAACTCCATCCTGATAAATGGACA |
| | | GTACAGCCTATAGTGCTGCCAGAAAAAGAC |
| | | AGCTGGACTGTCAATGACATACAGAAGTTA |
| | | GTGGGGAAATTGAATTGGGCAAGTCAGATT |
| | | TACCCAGGGATTAAAGTAAGGCAATTATGT |
| | | AAACTCCTTAGAGGAACCAAAGCACTAACA |
| | | GAAGTAATACCACTAACAGAAGAAGCAGAG |
| | | CTAGAACTGGCAGAAAACAGAGAGATTCTA |
| | | AAAGAACCAGTACATGGAGTGTATTATGAC |
| | | CCATCAAAAGACTTAATAGCAGAAATACAG |
| | | AAGCAGGGGCAAGGCCAATGGACATATCAA |
| | | ATTTATCAAGAGCCATTTAAAAATCTGAAA |
| | | ACAGGAAAATATGCAAGAATGAGGGGTGCC |
| | | CACACTAATGATGTAAAACAATTAACAGAG |
| | | GCAGTGCAAAAAATAACCACAGAAAGCATA |
| | | GTAATATGGGGAAAGACTCCTAAATTTAAA |
| | | CTGCCCATACAAAAGGAAACATGGGAAACA |
| | | TGGTGGACAGAGTATTGGCAAGCCACCTGG |
| | | ATTCCTGAGTGGGAGTTTGTTAATACCCCT |
| | | CCCTTAGTGAAATTATGGTACCAGTTAGAG |
| | | AAAGAACCCATAGTAGGAGCAGAAACCTTC |
| | | TATGTAGATGGGGCAGCTAACAGGGAGACT |
| | | AAATTAGGAAAAGCAGGATATGTTACTAAT |
| | | AGAGGAAGACAAAAAGTTGTCACCCTAACT |
| | | GACACAACAAATCAGAAGACTGAGTTACAA |
| | | GCAATTTATCTAGCTTTGCAGGATTCGGGA |
| | | TTAGAAGTAAACATAGTAACAGACTCACAA |
| | | TATGCATTAGGAATCATTCAAGCACAACCA |
| | | GATCAAAGTGAATCAGAGTTAGTCAATCAA |
| | | ATAATAGAGCAGTTAATAAAAAAGGAAAAG |
| | | GTCTATCTGGCATGGGTACCAGCACACAAA |
| | | GGAATTGGAGGAAATGAACAAGTAGATAAA |
| | | TTAGTCAGTGCTGGAATCAGGAAAGTACTA |
| | | tctggcggctcaaaaagaaccgccgacggc |
| | | agcgaattcgagcccaagaagaagaggaaa |
| | | gtc |
| | | (SEQ ID NO: 36) |
| V22 | PISPIETVPVKLKPGMDGPKVKQWPLTEEK | CCCATTAGCCCTATTGAGACTGTACCAGTA |
| | IKALVEICTEMEKEGKISKIGPENPYNTPV | AAATTAAAGCCAGGAATGGATGGCCCAAAA |
| | FAIKKKDSTKWRKLVDFRELNKRTQDFWEV | GTTAAACAATGGCCATTGACAGAAGAAAAA |
| | QLGIPHPAGLKKKKSVTVLDVGDAYFSVPL | ATAAAAGCATTAGTAGAAATTTGTACAGAG |
| | DEDFRKYTAFTIPSINNETPGIRYQYNVLP | ATGGAAAAGGAAGGGAAAATTTCAAAAATT |
| | QGWKGSPAIFQSSMTKILEPFRKQNPDIVI | GGGCCTGAAAATCCATACAATACTCCAGTA |
| | YQYMDDLYVGSDLEIGQHRTKIEELRQHLL | TTTGCCATAAAGAAAAAAGACAGTACTAAA |
| | RWGLTTPDKKHQKEPPFLWMGYELHPDKWT | TGGAGAAAATTAGTAGATTTCAGAGAACTT |
| | VQPIVLPEKDSWTVNDIQKLVGKLNWASQI | AATAAGAGAACTCAAGACTTCTGGGAAGTT |
| | YPGIKVRQLCKLLRGTKALTEVIPLTEEAE | CAATTAGGAATACCACATCCCGCAGGGTTA |
| | LELAENREILKEPVHGVYYDPSKDLIAEIQ | AAAAAGAAAAAATCAGTAACAGTACTGGAT |
| | KQGQGQWTYQIYQEPFKNLKTGKYARMRGA | GTGGGTGATGCATATTTTTCAGTTCCCTTA |
| | HTNDVKQLTEAVQKITTESIVIWGKTPKFK | GATGAAGACTTCAGGAAGTATACTGCATTT |
| | LPIQKETWETWWTEYWQATWIPEWEFVNTP | ACCATACCTAGTATAAACAATGAGACACCA |
| | PLVKLWYQLEKEPIVGAET | GGGATTAGATATCAGTACAATGTGCTTCCA |
| | (SEQ ID NO: 17) | CAGGGATGGAAAGGATCACCAGCAATATTC |
| | | CAAAGTAGCATGACAAAAATCTTAGAGCCT |
| | | TTTAGAAAACAAAATCCAGACATAGTTATC |
| | | TATCAATACATGGATGATTTGTATGTAGGA |
| | | TCTGACTTAGAAATAGGGCAGCATAGAACA |
| | | AAAATAGAGGAGCTGAGACAACATCTGTTG |
| | | AGGTGGGGACTTACCACACCAGACAAAAAA |
| | | CATCAGAAAGAACCTCCATTCCTTTGGATG |
| | | GGTTATGAACTCCATCCTGATAAATGGACA |
| | | GTACAGCCTATAGTGCTGCCAGAAAAAGAC |
| | | AGCTGGACTGTCAATGACATACAGAAGTTA |
| | | GTGGGGAAATTGAATTGGGCAAGTCAGATT |
| | | TACCCAGGGATTAAAGTAAGGCAATTATGT |
| | | AAACTCCTTAGAGGAACCAAAGCACTAACA |
| | | GAAGTAATACCACTAACAGAAGAAGCAGAG |
| | | CTAGAACTGGCAGAAAACAGAGAGATTCTA |
| | | AAAGAACCAGTACATGGAGTGTATTATGAC |
| | | CCATCAAAAGACTTAATAGCAGAAATACAG |
| | | AAGCAGGGGCAAGGCCAATGGACATATCAA |
| | | ATTTATCAAGAGCCATTTAAAAATCTGAAA |
| | | ACAGGAAAATATGCAAGAATGAGGGGTGCC |
| | | CACACTAATGATGTAAAACAATTAACAGAG |
| | | GCAGTGCAAAAAATAACCACAGAAAGCATA |
| | | GTAATATGGGGAAAGACTCCTAAATTTAAA |
| | | CTGCCCATACAAAAGGAAACATGGGAAACA |
| | | TGGTGGACAGAGTATTGGCAAGCCACCTGG |
| | | ATTCCTGAGTGGGAGTTTGTTAATACCCCT |
| | | CCCTTAGTGAAATTATGGTACCAGTTAGAG |
| | | AAAGAACCCATAGTAGGAGCAGAAACCtct |
| | | ggcggctcaaaaagaaccgccgacggcagc |
| | | gaattcgagcccaagaagaagaggaaagtc |
| | | (SEQ ID NO: 37) |
| V23 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLFSTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFTA | TTCCAATGGAAAGTCCTGCCTCAGGGTATG |
| | ASSST | ACTTGCAGCCCAACAATTTGCCAGCTTATT |
| | (SEQ ID NO: 18) | GTGGGACAAATCCTTGAACCCTTGAGACTT |
| | | AAACACCCAAGTCTCAGGATGCTCCACTAC |
| | | ATGGACGACCTGCTTCTTGCTGCATCCAGC |
| | | CATGATGGACTTGAGGCCGCCGGCGAAGAA |
| | | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAGcT |
| | | GCTTCATCTTCAACCtctggcggctcaaaa |
| | | agaaccgccgacggcagcgaattcgagccc |
| | | aagaagaagaggaaagtc |
| | | (SEQ ID NO: 38) |
| V24 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLFSTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFTA | TTCCAATGGAAAGTCCTGCCTCAGGGTATG |
| | ASSSTHKGVVVWREGPRWEIKEIADLGASV | ACTTGCAGCCCAACAATTTGCCAGCTTATT |
| | QQLAARAVA | GTGGGACAAATCCTTGAACCCTTGAGACTT |
| | (SEQ ID NO: 19) | AAACACCCAAGTCTCAGGATGCTCCACTAC |
| | | ATGGACGACCTGCTTCTTGCTGCATCCAGC |
| | | CATGATGGACTTGAGGCCGCCGGCGAAGAA |
| | | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAGcT |
| | | GCTTCATCTTCAACCCACAAAGGCGTAGTG |
| | | GTTTGGCGAGAGGGCCCCAGGTGGGAGATA |
| | | AAAGAAATAGCCGACCTCGGGGCAAGCGTT |
| | | CAACAGCTTGcGGCCAGAGCAGTGGCTtct |
| | | ggcggctcaaaaagaaccgccgacggcagc |
| | | gaattcgagcccaagaagaagaggaaagtc |
| | | (SEQ ID NO: 39) |
| V25 | TVALHLAIPLKWKPNHTPVWIDQWPLPEGK | ACTGTGGCTCTGCATCTGGCCATCCCCCTT |
| | LVALTQLVEKELQLGHIEPSLSCWNTPVFV | AAGTGGAAACCCAATCATACACCAGTGTGG |
| | IRKASGSYRLLHDLRAVNAKLVPFGAVQQG | ATTGACCAATGGCCCCTCCCCGAGGGGAAG |
| | APVLSALPRGWPLMVLDLKDCFFSIPLAEQ | CTGGTAGCCCTGACACAGCTGGTCGAGAAG |
| | DREAFAFTLPSVNNQAPARRFQWKVLPQGM | GAGCTCCAGCTCGGGCATATCGAACCCTCT |
| | TCSPTICQLIVGQILEPLRLKHPSLRMLHY | TTGTCCTGTTGGAACACACCTGTCTTTGTC |
| | MDDLLLAASSHDGLEAAGEEVISTLERAGF | ATTAGAAAGGCAAGTGGCTCCTATCGCCTC |
| | TISPDKVQREPGVQYLGYKLGSTYVAPVGL | TTGCACGATTTGAGAGCCGTGAACGCCAAA |
| | VAEPRIATLWDVQKLVGSLQWLRPALGIPP | CTCGTACCCTTCGGTGCAGTGCAGCAAGGT |
| | RLMGPFYEQLRGSDPNEAREWNLDMKMAWR | GCCCCCGTATTGAGCGCTCTTCCTCGCGGA |
| | EIVQLSTTAALERWDPALPLEGAVARCEQG | TGGCCTCTGATGGTACTCGACCTCAAAGAC |
| | AIGVLGQGLSTHPRPCLWLFSTQPTKAFTA | TGTTTTTTCAGTATACCCCTCGCCGAGCAA |
| | WLEVLTLLITKLRASAVRTFGKEVDILLLP | GACAGAGAGGCATTCGCCTTCACCTTGCCA |
| | ACFREDLPLPEGILLALRGFAGKIRSSDTP | TCCGTAAATAATCAGGCCCCAGCCCGGAGG |
| | SIFDIARPLHVSLKVRVTDHPVPGPTVFTA | TTCCAATGGAAAGTCCTGCCTCAGGGTATG |
| | ASSSTHKGVVVWREGPRWEIKEIADLGASV | ACTTGCAGCCCAACAATTTGCCAGCTTATT |
| | QQLAARAVAMALLLWPTTPTNVVTASAFVA | GTGGGACAAATCCTTGAACCCTTGAGACTT |
| | KMLLKMGQEGVPS | AAACACCCAAGTCTCAGGATGCTCCACTAC |
| | (SEQ ID NO: 20) | ATGGACGACCTGCTTCTTGCTGCATCCAGC |
| | | CATGATGGACTTGAGGCCGCCGGCGAAGAA |
| | | GTTATATCAACTTTGGAACGCGCTGGATTC |
| | | ACTATATCACCAGATAAAGTACAGCGGGAG |
| | | CCTGGGGTGCAGTATCTGGGTTACAAGCTC |
| | | GGCTCCACATATGTCGCCCCTGTCGGACTG |
| | | GTGGCCGAGCCTAGAATCGCAACACTGTGG |
| | | GACGTTCAAAAATTGGTAGGGTCACTGCAA |
| | | TGGTTGAGACCTGCCCTCGGCATTCCCCCC |
| | | CGGTTGATGGGACCATTTTACGAACAATTG |
| | | CGGGGAAGCGATCCAAACGAAGCACGAGAG |
| | | TGGAATCTGGATATGAAAATGGCCTGGCGC |
| | | GAAATTGTACAGCTGAGTACAACAGCAGCA |
| | | TTGGAACGATGGGACCCCGCTCTGCCTCTT |
| | | GAAGGAGCTGTGGCCCGATGCGAGCAGGGA |
| | | GCTATCGGTGTCCTTGGCCAAGGGTTGAGC |
| | | ACTCACCCTCGACCATGTTTGTGGTTGTTT |
| | | AGCACTCAGCCTACTAAAGCATTTACCGCC |
| | | TGGCTCGAGGTCCTCACATTGTTGATAACC |
| | | AAACTTCGCGCAAGCGCAGTGCGGACCTTC |
| | | GGAAAAGAAGTCGACATTCTGTTGCTCCCT |
| | | GCCTGTTTCAGGGAAGACCTTCCATTGCCC |
| | | GAGGGCATATTGCTGGCCCTCAGAGGCTTC |
| | | GCTGGAAAGATCCGAAGTTCAGACACCCCA |
| | | TCCATCTTCGATATAGCACGCCCTCTGCAT |
| | | GTATCCCTTAAAGTGAGGGTAACTGACCAC |
| | | CCTGTACCCGGACCCACCGTCTTCACAGcT |
| | | GCTTCATCTTCAACCCACAAAGGCGTAGTG |
| | | GTTTGGCGAGAGGGCCCCAGGTGGGAGATA |
| | | AAAGAAATAGCCGACCTCGGGGCAAGCGTT |
| | | CAACAGCTTGcGGCCAGAGCAGTGGCTATG |
| | | GCCTTGCTGCTGTGGCCTACTACTCCCACC |
| | | AATGTTGTGACAGcTTCTGCCTTCGTCGCA |
| | | AAGATGTTGCTGAAGATGGGCCAAGAGGGC |
| | | GTTCCTTCAtctggcggctcaaaaagaacc |
| | | gccgacggcagcgaattcgagcccaagaag |
| | | aagaggaaagtc |
| | | (SEQ ID NO: 40) |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A polypeptide comprising:
a single subunit of a multi-subunit reverse transcriptase, or a variant or fragment thereof, linked to a sequence-specific nuclease, or a variant or active fragment thereof,
wherein the single subunit of a multi-subunit reverse transcriptase, or a variant or fragment thereof comprises less than 800 amino acids.

2. The polypeptide of claim 1, wherein the single subunit of a multi-subunit reverse transcriptase, or a variant or fragment thereof comprises an RNaseH domain.

3. The polypeptide of claim 2, wherein the RNaseH domain is partially or completely inactive or removed.

4. The polypeptide of claim 1, wherein the single subunit of a multi-subunit reverse transcriptase, or a variant or fragment thereof comprises less than 600 amino acids.

5. The polypeptide of claim 1, wherein the single subunit of a multi-subunit reverse transcriptase, or a variant or fragment thereof comprises a connection subdomain.

6. The polypeptide of claim 1, wherein the single subunit of a multi-subunit reverse transcriptase, or a variant or fragment thereof is derived from: avian myeloblastosis virus reverse transcriptase (AMV RT)-alpha subunit, Rous sarcoma virus Transcriptase (RSV RT)-alpha subunit, or HIV-1 reverse transcriptase (RT) p66 subunit.

7. The polypeptide of claim 1, wherein the single subunit of a multi-subunit reverse transcriptase, or a variant or fragment thereof comprises an amino acid sequence having at least 70% identity to any of SEQ ID NOs: 4, 8, 9, 14, or 16.

8. The polypeptide of claim 1, wherein the single subunit of a multi-subunit reverse transcriptase, or a variant or fragment thereof is linked to the C terminus of the sequence-specific nuclease, or a variant or active fragment thereof.

9. The polypeptide of claim 1, wherein the sequence-specific nuclease is Cas9 or a variant or fragment thereof.

10. A system for modifying a target nucleic acid comprising:
a polypeptide of claim 1, or a nucleic acid encoding thereof; and
one or more RNA polynucleotides comprising a spacer sequence and an extension sequence comprising a primer binding sequence (PBS) and a reverse transcriptase template (RTT) sequence, or one or more nucleic acids encoding thereof.

11. The system of claim 10, wherein the spacer sequence and the extension sequence are contained within a single RNA polynucleotide.

12. The system of claim 10, further comprising:
a nicking guide RNA, or a nucleic acid encoding thereof; and/or
a target nucleic acid.

13. A method for modifying a target nucleic acid comprising contacting the target nucleic acid with:
a polypeptide of claim 1; and
one or more RNA polynucleotides comprising a spacer sequence and an extension sequence comprising a primer binding sequence (PBS) and a reverse transcriptase template (RTT) sequence, wherein the RTT sequence encodes one or more nucleotides to modify the target nucleic acid; and
optionally a nicking guide RNA (ngRNA).

14. The method of claim 13, wherein the RTT sequence encodes one or more nucleotide substitutions, additions, or deletions to correct or confer a disease-causing mutation in the target nucleic acid.

15. The method of claim 13, wherein the target nucleic acid is in a cell.
